(19) United States
(12) Patent Application Publication
(10) Pub. No.: US 2007/0032473 A1
(43) Pub. Date: Feb. 8, 2007

(54) Substituted amides and their use as medicaments

(76) Inventors: Kai Gerlach, Biberach (DE); Henning Priepke, Warthausen (DE); Roland Pfau, Biberach (DE); Wolfgang Wienen, Biberach (DE); Annette Schuler-Metz, Ulm (DE); Georg Dahmann, Attenweiler (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/485,152

(22) Filed: Jul. 12, 2006

(30) Foreign Application Priority Data
Jul. 19, 2005 (EP) 05 015 588

(51) Int. Cl. A61K 31/55; C07D 487/02

(52) U.S. Cl. 514/215; 540/568

(57) ABSTRACT

Substituted amides of formula (I) 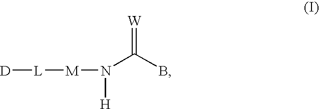 wherein D, L, M, W, and B are defined as in the specification, or a tautomer, enantiomer, or salt thereof, particularly the physiologically acceptable salts thereof with inorganic or organic acids or bases, which have valuable properties.

### RELATED APPLICATIONS

This application claims priority to European Patent Application 05 015 588.6, filed Jul. 19, 2005, which is hereby incorporated by reference in its entirety.

### FIELD OF THE INVENTION

The present invention relates to new substituted amides of general formula (I) 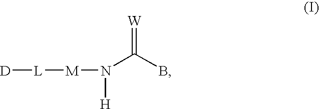
the tautomers, the enantiomers, the diastereomers, the mixtures, and the salts thereof, particularly the physiologically acceptable salts thereof with inorganic or organic acids or bases, which have valuable properties.

The compounds of the above general formula (I) and the tautomers, the enantiomers, the diastereomers, the mixtures, and the salts thereof, particularly the physiologically acceptable salts thereof with inorganic or organic acids or bases, and the stereoisomers thereof have valuable pharmacological properties, particularly an antithrombotic activity and a factor Xa-inhibiting activity.

The present application relates to new compounds of the above general formula (I), the preparation thereof, the pharmaceutical compositions containing the pharmacologically effective compounds, the preparation, and use thereof.

A first embodiment of the present invention includes those compounds of general formula (I), wherein:

D denotes a substituted bicyclic ring system of formula (II) 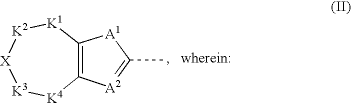
K¹ and K⁴ each independently of one another represent a —CH₂—, —CHR^{7a}—, —CR^{7b}R^{7c}—, or a —C(O)— group, wherein R^{7a}/R^{7b}/R^{7c} each independently of one another represent a fluorine atom, a hydroxy, C₁₋₅-alkoxy, amino, C₁₋₅-alkylamino, di-(C₁₋₅-alkyl)-amino, C₃₋₅-cycloalkyleneimino, C₁₋₅-alkylcarbonylamino group, a C₁₋₅-alkyl group which may be substituted by one to three fluorine atoms, a hydroxy-C₁₋₅-alkyl, C₁₋₅-alkoxy-C₁₋₅-alkyl, amino-C₁₋₅-alkyl, C₁₋₅-alkylamino-C₁₋₅-alkyl, di-(C₁₋₅-alkyl)-amino-C₁₋₅-alkyl, C₄₋₇-cycloalkyleneimino-C₁₋₅-alkyl, carboxy-C₀₋₅-alkyl, C₁₋₅-alkoxycarbonyl-C₀₋₅-alkyl, aminocarbonyl-C₀₋₅-alkyl, C₁₋₅-alkylaminocarbonyl-C₀₋₅-alkyl, di-(C₁₋₅-alkyl)-aminocarbonyl-C₀₋₅-alkyl, or C₄₋₇-cycloalkyleneiminocarbonyl-C₀₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom may form a 3-, 4-, 5-, 6-, or 7-membered saturated carbocycle, or a cyclopentene, cyclohexene, oxetan, azetidine, thietan, tetrahydrofuran, pyrrolidine, tetrahydrothiophene, tetrahydropyran, piperidine, pentamethylene sulfide, hexamethyleneimnine, 1,3-dioxolane, 1,4-dioxane, hexahydropyridazine, piperazine, thiomorpholine, morpholine, 2-imidazolidinone, 2-oxazolidinone, tetrahydro-2(1H)-pyrimidinone, or [1,3]oxazinan-2-one ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides an N atom may be replaced by a —C(O)— group, and/or the imino groups of which may each be substituted by a C₁₋₃-alkyl or C₁₋₃-alkylcarbonyl group, and/or wherein the sulfur atom may be oxidized to form a sulfoxide or sulfone group,
K² and K³ each independently of one another represent a —CH₂—, CHR^{8a}—, —CR^{8b}R^{8c}—, or a —C(O)— group, wherein R^{8a}/R^{8b}/R^{8c} each independently of one another represent a C₁₋₅-alkyl group which may be substituted by one to three fluorine atoms, a hydroxy-C₁₋₅-alkyl, C₁₋₅-alkoxy-C₁₋₅-alkyl, amino-C₁₋₅-alkyl, C₁₋₅-alkyl-amino-C₁₋₅-alkyl, di-(C₁₋₅-alkyl)-amino-C₁₋₅-alkyl, C₄₋₇-cyclo-alkyleneimino-C₁₋₅-alkyl, carboxy-C₁₋₅-alkyl, C₁₋₅-alkoxycarbonyl-C₁₋₅-alkyl, aminocarbonyl-C₁₋₅-alkyl, C₁₋₅-alkylaminocarbonyl-C₁₋₅-alkyl, di-(C₁₋₅-alkyl)-aminocarbonyl-C₁₋₅-alkyl, or C₄₋₇-cycloalkyleneiminocarbonyl-C₁₋₅-alkyl group, or two groups R^{8b}/R^{8c} together with the cyclic carbon atom may form a 3-, 4-, 5-, 6-, or 7-membered saturated carbocycle or a cyclopentene, cyclohexene, oxetan, azetidine, thietan, tetrahydrofuran, pyrrolidine, tetrahydrothiophene, tetrahydropyran, piperidine, pentamethylene sulfide, hexamethyleneimine, hexahydropyridazine, tetrahydro-2(1H)-pyrimidinone, or [1,3]oxazinan-2-one ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides a nitrogen atom may be replaced by a —CO— group, and/or the imino groups of which may each be substituted by a C₁₋₃-alkyl or C₁₋₃-alkylcarbonyl group, and/or wherein the sulfur atom may be oxidized to form a sulfoxide or sulfone group, with the proviso that a heteroatom introduced by R^{8b} or R^{8c} cannot be separated from X in formula I by only one carbon atom, and
   in total formula (II) should contain a maximum of four groups selected from among R^{7a}, R^{7b}, R^{7c}, R^{8a}, R^{8b}, and R^{8c},
X denotes an oxygen or sulfur atom, a sulfene, sulfone, or —N(R¹)— group, wherein R¹ denotes a hydrogen atom or a hydroxy, C₁₋₃-alkoxy, amino, C₁₋₃-alkylamino, di-(C₁₋₃-alkyl)-amino, C₁₋₅-alkyl, C₃₋₅-alkenyl-CH₂, C₃₋₅-alkynyl-CH₂, C₃₋₆-cycloalkyl, C₄₋₆-cycloalkenyl, oxetan-3-yl, tetrahydrofuran-3-yl, benzyl, C₁₋₅-alkylcarbonyl, trifluoromethylcarbonyl, C₃₋₆-cycloalkylcarbonyl, C₁₋₅-alkylsulfonyl, C₃₋₆-cycloalkylsulfonyl, aminocarbonyl, C₁₋₅-alkylaminocarbonyl, di-(C₁₋₅-alkyl)-aminocarbonyl, C₁₋₅-alkyloxycarbonyl, or C₄₋₇-cycloalkyleneiminocarbonyl group, wherein the methylene and methyl groups present in the groups mentioned previously may additionally be substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group, as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes an oxygen or sulfur atom, a —C(R¹⁰)═N—, —N═C(R¹⁰)—, or —C(R¹⁰)═C(R¹¹)— group,
A² denotes either a nitrogen atom or a ═C(R¹²)— group, wherein R¹⁰, R¹¹, and R¹² each independently of one another represent a hydrogen, fluorine, chlorine, bromine, or iodine atom, or a C₁₋₅-alkyl, —CF₃, C₂₋₅-alkenyl, C₂₋₅-alkynyl, cyano, carboxy, C₁₋₅-alkoxycarbonyl, hydroxy, C₁₋₃-alkoxy, CF₃O, CHF₂O, CH₂FO, amino, C₁₋₅-alkylamino, di-(C₁₋₅-alkyl)-amino, or C₄₋₇-cycloalkyleneimino group,
L denotes a substituted ring system of formula (IIa) or (IIb) 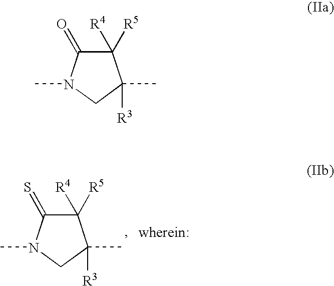
R³ denotes a hydrogen atom or a C₁₋₃-alkyl group,
R⁴ and R⁵ each independently of one another represent a hydrogen atom, a hydroxy group, an —OR⁹ group, a C₂₋₆-alkenyl, or C₂₋₆-alkynyl group,
   a straight-chain or branched C₁₋₆-alkyl group, wherein the hydrogen atoms of the straight-chain or branched C₁₋₆-alkyl group may optionally be wholly or partly replaced by fluorine atoms, and wherein the straight-chain or branched C₁₋₆-alkyl group may optionally be substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c} group, and additionally a methylene group adjacent to an abovementioned —NR^{8c} group may be replaced by a carbonyl group,
   a phenyl or heteroaryl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a phenyl-C₁₋₅-alkyl or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which may optionally be substituted in the C₁₋₅-alkyl moiety by a hydroxy or a C₁₋₅-alkyloxy group, wherein the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group,
   a 3- to 7-membered cycloalkyl, cycloalkyleneimino, cycloalkyl-C₁₋₅-alkyl, or cycloalkyleneimino-C₁₋₃-alkyl group, wherein in 4- to 7-membered cyclic groups in the cyclic moiety a methylene group may optionally be replaced by a —N(R^{8c}) group, an oxygen or sulfur atom, or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyleneimino, cycloalkyl-C₁₋₅-alkyl, or cycloalkyleneimino-C₁₋₃-alkyl group as hereinbefore defined wherein two heteroatoms selected from among oxygen and nitrogen are separated from one another by precisely one optionally substituted —CH₂ group, is excluded, wherein a 3- to 7-membered cycloalkyl, cycloalkyleneimino, cycloalkyl-C₁₋₅-alkyl, or cycloalkyleneimino-C₁₋₃-alkyl group as hereinbefore defined may be substituted at one or two —CH₂ groups by one or two C₁₋₃-alkyl groups in each case,
   with the proviso that R⁴ and R⁵ cannot simultaneously be defined as hydroxy or —OR⁹ groups, and wherein:
   R⁹ denotes a straight-chain or branched C₁₋₆-alkyl group, wherein the hydrogen atoms of the straight-chain or branched C₁₋₆alkyl group may optionally be wholly or partly replaced by fluorine atoms, and wherein the straight-chain or branched C₁₋₆-alkyl group may optionally be substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c}— group, and additionally a methylene group adjacent to an abovementioned —NR^{8c}— group may be replaced by a carbonyl group, with the proviso that the replacement of hydrogen atoms of the first carbon atom of the straight-chain or branched C₁₋₆-alkyl group by substituents selected from among oxygen, sulfur, or nitrogen is excluded,
   a phenyl, heteroaryl, phenyl-C₁₋₅-alkyl, or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a 3- to 7-membered cycloalkyl, cycloalkyl-C₁₋₅-alkyl, or cycloalkyleneimino-C₂₋₃-alkyl group, wherein in 4- to 7-membered cyclic groups in the cyclic moiety a methylene group may optionally be replaced by a —N(R^{8c})— group, an oxygen or sulfur atom, or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b}) or —N(R^{8b})C(O)N(R^{8b}) or —N(R^{8b})S(O)₂N(R^{8b})— group, with the proviso that a 3- to 7-membered cycloalkyl, cycloalkyl-C₁₋₅-alkyl, or cycloalkyleneimino-C₂₋₃-alkyl group as hereinbefore defined wherein two heteroatoms selected from among oxygen and nitrogen are separated from one another by precisely one optionally substituted —CH₂— group, is excluded, wherein a 3- to 7-membered cycloalkyl, cycloalkyl-C₁₋₅-alkyl, or cycloalkyleneimino-C₂₋₃-alkyl group as hereinbefore defined may be substituted at one or two —CH₂— groups by in each case one or two C₁₋₃-alkyl groups, or
R⁴ and R⁵ together with the carbon atom to which they are bound, form a C₃₋₈-cycloalkyl or C₃₋₈-cycloalkenyl group, wherein one of the methylene groups of a C₄₋₈-cycloalkyl group may be replaced by an oxygen or sulfur atom or a —N(R^{8c})—, carbonyl, sulfinyl, or sulfonyl group, and/or two directly adjacent methylene groups of a C₄₋₈-cycloalkyl group may together be replaced by a —C(O)N(R^{8b})— or —S(O)₂N(R^{8b})— group, and/or three directly adjacent methylene groups of a C₆₋₈-cycloalkyl group may together be replaced by an —OC(O)N(R^{8b})—, —N(R^{8b})C(O)N(R^{8b})—, or —N(R^{8b})S(O)₂N(R^{8b})— group, wherein one to three carbon atoms of a C₃₋₈-cycloalkyl group may optionally be substituted independently of one another by in each case one or two identical or different halogen atoms, or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino groups, wherein 1 to 2 carbon atoms of a C₃₋₈-cycloalkenyl group may optionally be substituted independently of one another by in each case a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, or C₃₋₆-cycloalkyleneiminosulfonyl group, and 1 to 2 carbon atoms of a C₄₋₈-cycloalkenyl group which are not bound to another carbon atom by a double bond, may optionally be substituted independently of one another by a fluorine atom or a hydroxy, C₁₋₅-alkyloxy, C₁₋₅-alkylcarbonyloxy, C₁₋₅-alkylsulfanyl, C₁₋₅-alkylsulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino group, with the proviso that a C₃₋₈-cycloalkyl or C₃₋₈-cycloalkenyl group of this kind, formed from R⁴ and R⁵ together, wherein two heteroatoms in the cyclic group selected from among oxygen and nitrogen are separated from one another by precisely one optionally substituted —CH₂— group, and/or wherein one or both methylene groups of the cyclic group which are directly connected to the carbon atom to which the groups R⁴ and R⁵ are bound are replaced by a heteroatom selected from among oxygen, nitrogen, and sulfur, and/or wherein a substituent bound to the cyclic group, which is characterized in that a heteroatom selected from among oxygen, nitrogen, and sulfur, and a halogen atom is bound directly to the cyclic group, is separated from another heteroatom selected from among oxygen, nitrogen, and sulfur, with the exception of the sulfone group, by precisely one, optionally substituted, methylene group, and/or wherein two oxygen atoms are joined together directly, is excluded,
M denotes a —CH₂—, —CHR³, —CR³R³— group, or a bond,
W denotes an oxygen or sulfur atom,
B denotes a thiophene ring according to formula (III) 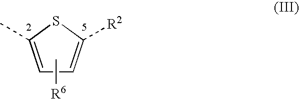
   which is bound to the carbonyl group in formula (I) via the 2-position and which is substituted in the 5-position by R² and optionally additionally by R⁶, wherein R² denotes a fluorine, chlorine, bromine, or iodine atom, or a methoxy, C₁₋₂-alkyl, or ethynyl group, and R⁶ denotes a hydrogen, fluorine, chlorine, bromine, or iodine atom, or a C₁₋₂-alkyl or amino group,
wherein, unless stated otherwise, by the term “heteroaryl group” mentioned hereinbefore in the definitions is meant a monocyclic 5- or 6-membered heteroaryl group, wherein:
   the 6-membered heteroaryl group contains one, two, or three nitrogen atoms, and
   the 5-membered heteroaryl group contains an imino group optionally substituted by a C₁₋₃-alkyl group, or an oxygen or sulfur atom, or an imino group optionally substituted by a C₁₋₃-alkyl group, or an oxygen or sulfur atom and additionally a nitrogen atom, or an imino group optionally substituted by a C₁₋₃-alkyl group and two or three nitrogen atoms,
   and moreover a phenyl ring optionally substituted by a fluorine, chlorine, or bromine atom, a C₁₋₃-alkyl, hydroxy, C₁₋₃-alkyloxy group, amino, C₁₋₃-alkylamino, di-(C₁₋₃-alkyl)-amino, or C₃₋₆-cycloalkyleneimino group may be fused to the abovementioned monocyclic heteroaryl groups via two adjacent carbon atoms,
   and the bond is effected via a nitrogen atom or a carbon atom of the heterocyclic moiety or a fused-on phenyl ring,
wherein, unless stated otherwise, by the term “halogen atom” mentioned hereinbefore in the definitions is meant an atom selected from among fluorine, chlorine, bromine, and iodine,
wherein the alkyl, alkenyl, alkynyl, and alkoxy groups contained in the previously mentioned definitions which have more than two carbon atoms may, unless stated otherwise, be straight-chain or branched and the alkyl groups in the previously mentioned dialkylated groups, for example, the dialkylamino groups, may be identical or different,
and the hydrogen atoms of the methyl or ethyl groups contained in the foregoing definitions, unless otherwise stated, may be wholly or partly replaced by fluorine atoms,
the tautomers, the enantiomers, the diastereomers, the mixtures, and the salts thereof.

Examples of monocyclic heteroaryl groups are the pyridyl, N-oxy-pyridyl, pyrazolyl, pyridazinyl, pyrimidinyl, pyrazinyl, [1,2,3]triazinyl, [1,3,5]triazinyl, [1,2,4]triazinyl, pyrrolyl, imidazolyl, [1,2,4]triazolyl, [1,2,3]triazolyl, tetrazolyl, furanyl, isoxazolyl, oxazolyl, [1,2,3]oxadiazolyl, [1,2,4]oxadiazolyl, furazanyl, thiophenyl, thiazolyl, isothiazolyl, [1,2,3]thiadiazolyl, [1,2,4]thiadiazolyl, or [1,2,5]thiadiazolyl group.

Examples of bicyclic heteroaryl groups are the benzimidazolyl, benzofuranyl, benzo[c]furanyl, benzothiophenyl, benzo[c]thiophenyl, benzothiazolyl, benzo[c]isothiazolyl, benzo[d]isothiazolyl, benzoxazolyl, benzo[c]isoxazolyl, benzo[d]isoxazolyl, benzo[1,2,5]oxadiazolyl, benzo[1,2,5]thiadiazolyl, benzo[1,2,3]thiadiazolyl, benzo[d][1,2,3]triazinyl, benzo[1,2,4]triazinyl, benzotriazolyl, cinnolinyl, quinolinyl, N-oxy-chinolinyl, isoquinolinyl, quinazolinyl, N-oxyquinazolinyl, quinoxalinyl, phthalazinyl, indolyl, isoindolyl, or 1-oxa-2,3-diazaindenyl group.

Examples of the C₁₋₆-alkyl groups mentioned hereinbefore in the definitions are the methyl, ethyl, 1-propyl, 2-propyl, n-butyl, sec-butyl, tert-butyl, 1-pentyl, 2-pentyl, 3-pentyl, neopentyl, 3-methyl-2-butyl, 1-hexyl, 2-hexyl, 3-hexyl, 3-methyl-2-pentyl, 4-methyl-2-pentyl, 3-methyl-3-pentyl, 2-methyl-3-pentyl, 2,2-dimethyl-3-butyl, or 2,3-dimethyl-2-butyl group.

Examples of the C₁₋₅-alkyloxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy, 3-pentyloxy, or neopentyloxy group.

Examples of the C₂₋₅-alkenyl groups mentioned hereinbefore in the definitions are the ethenyl, 1-propen-1-yl, 2-propen-1-yl, 1-buten-1-yl, 2-buten-1-yl, 3-buten-1-yl, 1-penten-1-yl, 2-penten-1-yl, 3-penten-1-yl, 4-penten-1-yl, 1-hexen-1-yl, 2-hexen-1-yl, 3-hexen-1-yl, 4-hexen-1-yl, 5-hexen-1-yl, but-1-en-2-yl, but-2-en-2-yl, but-1-en-3-yl, 2-methylprop-2-en-1-yl, pent-1-en-2-yl, pent-2-en-2-yl, pent-3-en-2-yl, pent4-en-2-yl, pent-1-en-3-yl, pent-2-en-3-yl, 2-methyl-but-1-en-1-yl, 2-methyl-but-2-en-1-yl, 2-methylbut-3-en-1-yl, or 2-ethylprop-2-en-1-yl- group.

Examples of the C₂₋₅-alkynyl groups mentioned hereinbefore in the definitions are the ethynyl, 1-propynyl, 2-propynyl, 1-butyn-1-yl, 1-butyn-3-yl, 2-butyn-1-yl, 3-butyn-1-yl, 1-pentyn-1-yl, 1-pentyn-3-yl, 1-pentyn-4-yl, 2-pentyn-1-yl, 2-pentyn-3-yl, 3-pentyn-1-yl, 4-pentyn-1-yl, 2-methyl-1-butyn-4-yl, 3-methyl-1-butyn-1-yl, or 3-methyl-1-butyn-3-yl group.

A second embodiment of the present invention encompasses those compounds of general formula (I), wherein:
D denotes a substituted bicyclic ring system of formula (II) 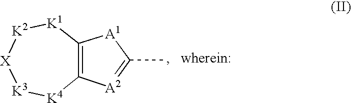
K¹ and K⁴ each independently of one another represent a —CH₂—, —CHR^{7a}—, —CR^{7b}R^{7c}—, or a —C(O)— group, wherein R^{7a}/R^{7b}/R^{7c} each independently of one another represent a fluorine atom, a hydroxy, C₁₋₅-alkoxy group, a C₁₋₅-alkyl group which may be substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides an N atom may be replaced by a —C(O)— group,
K² and K³ each independently of one another represent a —CH₂—, —CHR^{8a}—, —CR^{8b}R^{8c}—, or a —C(O)— group, wherein R^{8a}/R^{8b}/R^{8c} each independently of one another represent a C₁₋₅-alkyl group which may be substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups may be substituted, and/or the methylene groups thereof, if they are not bound to a heteroatom, may be substituted by one or two fluorine atoms, and/or wherein a —CH₂— group besides a nitrogen atom may be replaced by a —C(O)— group, with the proviso that a heteroatom introduced by R^{8b} or R^{8c} cannot be separated from X in formula I by only one carbon atom, and
   in total formula (II) should contain a maximum of four groups selected from among R^{7a}, R^{7b}, R^{7c}, R^{8a}, R^{8b}, and R^{8c},
X denotes an oxygen or sulfur atom, a sulfene, sulfone, or an —N(R⁸)— group, wherein R¹ denotes a hydrogen atom or a hydroxy, C₁₋₃-alkoxy, amino, C₁₋₃-alkylamino, di-(C₁₋₃-alkyl)-amino, C₁₋₅-alkyl, C₃₋₅-alkenyl-CH₂, C₃₋₅-alkynyl-CH₂, C₃₋₆-cycloalkyl, C₄₋₆-cycloalkenyl, oxetan-3-yl, tetrahydrofuran-3-yl, benzyl, C₁₋₅-alkylcarbonyl, trifluoromethylcarbonyl, C₃₋₆-cycloalkylcarbonyl, C₁₋₅-alkylsulfonyl, C₃₋₆-cycloalkylsulfonyl, aminocarbonyl, C₁₋₅-alkylaminocarbonyl, di-(C₁₋₅-alkyl)-aminocarbonyl, C₁₋₅-alkyloxycarbonyl, or C₄₋₇-cycloalkyleneiminocarbonyl group, wherein the methylene and methyl groups present in the groups mentioned previously may additionally be substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes an oxygen or sulfur atom, a —C(R¹⁰)═N—, —N═C(R¹⁰)—, or —C(R¹⁰)═C(R¹¹)— group,
A² denotes either a nitrogen atom or a ═C(R¹²)— group, wherein R¹⁰, R¹¹, and R¹² each independently of one another represent a hydrogen, fluorine, chlorine, bromine, or iodine atom, or a C₁₋₅-alkyl, —CF₃, C₂₋₅-alkenyl, C₂₋₅-alkynyl, cyano, carboxy, C₁₋₅-alkyoxycarbonyl, hydroxy, C₁₋₃-alkoxy, CF₃O, CHF₂O, CH₂FO, amino, C₁₋₅-alkylamino, di-(C₁₋₅-alkyl)-amino, or C₄₋₇-cycloalkyleneimino group,
L denotes a substituted ring system of formula (IIa) or (IIb) 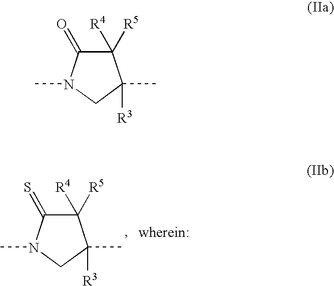
R³ denotes a hydrogen atom or a methyl group,
R⁴ and R⁵ each independently of one another represent a hydrogen atom, a hydroxy group, an —OR⁹ group, a C₂₋₆-alkenyl, or C₂₋₆-alkynyl group,
   a straight-chain or branched C₁₋₆-alkyl group, wherein the hydrogen atoms of the straight-chain or branched C₁₋₆-alkyl group may optionally be wholly or partly replaced by fluorine atoms, and wherein the straight-chain or branched C₁₋₆-alkyl group may optionally be substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c} group, and additionally a methylene group adjacent to an abovementioned —NR^{8c} group may be replaced by a carbonyl group,
   a phenyl or heteroaryl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a phenyl-C₁₋₅-alkyl or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which may optionally be substituted in the C₁₋₅-alkyl moiety by a hydroxy or a C₁₋₅-alkyloxy group, wherein the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group,
   a 3- to 7-membered cycloalkyl, cycloalkyleneimino, cycloalkyl-C₁₋₅-alkyl, or cycloalkyleneimino-C₁₋₃-alkyl group, wherein in 4- to 7-membered cyclic groups in the cyclic moiety a methylene group may optionally be replaced by an —N(R^{8c})— group, an oxygen or sulfur atom, or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyleneimino, cycloalkyl-C₁₋₅-alkyl, or cycloalkyleneimino-C₁₋₃-alkyl group as hereinbefore defined wherein two heteroatoms selected from among oxygen and nitrogen are separated from one another by precisely one optionally substituted —CH₂— group, is excluded, wherein a 3- to 7-membered cycloalkyl, cycloalkyleneimino, cycloalkyl-C₁₋₅-alkyl, or cycloalkyleneimino-C₁₋₃-alkyl group as hereinbefore defined may be substituted at one or two —CH₂ groups by one or two C₁₋₃-alkyl groups in each case,
   with the proviso that R⁴ and R⁵ cannot simultaneously be defined as hydroxy or —OR⁹ groups, and wherein:
   R⁹ denotes a straight-chain or branched C₁₋₆-alkyl group, wherein the hydrogen atoms of the straight-chain or branched C₁₋₆alkyl group may optionally be wholly or partly replaced by fluorine atoms, and wherein the straight-chain or branched C₁₋₆-alkyl group may optionally be substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl or —NR^{8c}— group, and additionally a methylene group adjacent to an abovementioned —NR^{8c}— group may be replaced by a carbonyl group, with the proviso that the replacement of hydrogen atoms of the first carbon atom of the straight-chain or branched C₁₋₆-alkyl group by substituents selected from among oxygen, sulfur, or nitrogen is excluded,
   a phenyl, heteroaryl, phenyl-C₁₋₅-alkyl, or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a 3- to 7-membered cycloalkyl, cycloalkyl-C₁₋₅-alkyl, or cycloalkyleneimino-C₂₋₃-alkyl group, wherein in 4- to 7-membered cyclic groups in the cyclic moiety a methylene group may optionally be replaced by a —N(R^{8c})— group, an oxygen or sulfur atom or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyl-C₁₋₅-alkyl, or cycloalkyleneimino-C₂₋₃-alkyl group as hereinbefore defined wherein two heteroatoms selected from among oxygen and nitrogen are separated from one another by precisely one optionally substituted —CH₂— group, is excluded, wherein a 3- to 7-membered cycloalkyl, cycloalkyl-C₁₋₅-alkyl, or cycloalkyleneimino-C₂₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group, wherein one of the methylene groups of a C₄₋₈-cycloalkyl group may be replaced by an oxygen or sulfur atom or a —N(R^{8c})—, carbonyl, sulfinyl, or sulfonyl group, and/or two directly adjacent methylene groups of a C₄₋₈-cycloalkyl group may together be replaced by a —C(O)N(R^{8b})— or —S(O)₂N(R^{8b})— group, and/or three directly adjacent methylene groups of a C₆₋₈-cycloalkyl group may together be replaced by an —OC(O)N(R^{8b}), —N(R^{8b})C(O)N(R^{8b})—, or —N(R^{8b})S(O)₂N(R^{8b})— group, wherein one to three carbon atoms of a C₃₋₈-cycloalkyl group may optionally be substituted independently of one another by in each case one or two identical or different halogen atoms, or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino groups, wherein 1 to 2 carbon atoms of a C₃₋₈-cycloalkenyl group may optionally be substituted independently of one another by in each case a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-arninosulfonyl, or C₃₋₆-cycloalkyleneiminosulfonyl group, and 1 to 2 carbon atoms of a C₄₋₈-cycloalkenyl group which are not bound to another carbon atom by a double bond, may optionally be substituted independently of one another by a fluorine atom or a hydroxy, C₁₋₅-alkyloxy, C₁₋₅-alkylcarbonyloxy, C₁₋₅-alkylsulfanyl, C₁₋₅-alkylsulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino group, with the proviso that a C₃₋₈-cycloalkyl or C₃₋₈-cycloalkenyl group of this kind, formed from R⁴ and R⁵ together, wherein two heteroatoms in the cyclic group selected from among oxygen and nitrogen are separated from one another by precisely one optionally substituted —CH₂— group, and/or wherein one or both methylene groups of the cyclic group which are directly connected to the carbon atom to which the groups R⁴ and R⁵ are bound are replaced by a heteroatom selected from among oxygen, nitrogen, and sulfur, and/or wherein a substituent bound to the cyclic group, which is characterized in that a heteroatom selected from among oxygen, nitrogen, and sulfur, and halogen atom is bound directly to the cyclic group, is separated from another heteroatom selected from among oxygen, nitrogen, and sulfur, with the exception of the sulfone group, by precisely one, optionally substituted, methylene group, and/or wherein two oxygen atoms are joined together directly, is excluded,
M denotes a —CH₂, —CHR³, —CR³R³— group, or a bond,
W denotes an oxygen or sulfur atom,
B denotes a thiophene ring according to formula (III) 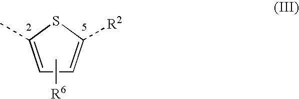
   which is bound to the carbonyl group in formula (1) via the 2-position and which is substituted in the 5-position by R² and optionally additionally by R⁶, wherein:
R² denotes a fluorine, chlorine, bromine, or iodine atom, or a methoxy, C₁₋₂-alkyl, or ethynyl group,
R⁶ denotes a hydrogen, fluorine, chlorine, bromine, or iodine atom, or a C₁₋₂-alkyl or amino group,
wherein, unless stated otherwise, by the term “heteroaryl group” mentioned hereinbefore in the definitions is meant a monocyclic 5- or 6-membered heteroaryl group, wherein:
   the 6-membered heteroaryl group contains one, two, or three nitrogen atoms, and
   the 5-membered heteroaryl group contains an imino group optionally substituted by a C₁₋₃-alkyl group or an oxygen or sulfur atom, or an imino group optionally substituted by a C₁₋₃-alkyl group or an oxygen or sulfur atom and additionally a nitrogen atom, or an imino group optionally substituted by a C₁₋₃-alkyl group and two or three nitrogen atoms,
   and moreover a phenyl ring optionally substituted by a fluorine, chlorine, or bromine atom, a C₁₋₃-alkyl, hydroxy, C₁₋₃-alkyloxy group, amino, C₁₋₃-alkylamino, di-(C₁₋₃-alkyl)-amino, or C₃₋₆-cycloalkyleneimino group may be fused to the abovementioned monocyclic heteroaryl groups via two adjacent carbon atoms,
   and the bond is effected via a nitrogen atom or a carbon atom of the heterocyclic moiety or a fused-on phenyl ring,
wherein, unless stated otherwise, by the term “halogen atom” mentioned hereinbefore in the definitions is meant an atom selected from among fluorine, chlorine, bromine, and iodine,
wherein the alkyl, alkenyl, alkynyl, and alkoxy groups contained in the previously mentioned definitions which have more than two carbon atoms may, unless stated otherwise, be straight-chain or branched and the alkyl groups in the previously mentioned dialkylated groups, for example, the dialkylamino groups, may be identical or different,
and wherein the hydrogen atoms of the methyl or ethyl groups contained in the foregoing definitions, unless otherwise stated, may be wholly or partly replaced by fluorine atoms,
the tautomers, the enantiomers, the diastereomers, the mixtures, and the salts thereof.

A third embodiment of the present invention encompasses those compounds of general formula (1), wherein:
D denotes a substituted bicyclic ring system of formula (II) 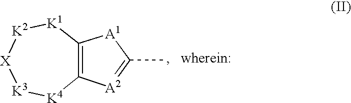
K¹ and K⁴ each independently of one another represent a —CH₂—, —CHR^{7a}—, CR^{7b}R^{7c}—, or a —C(O)— group, wherein R^{7a}/R^{7b}/R^{7c} each independently of one another represent a fluorine atom, a hydroxy, C₁₋₅-alkoxy group, a C₁₋₅-alkyl group which may be substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides an N atom may be replaced by a —C(O)— group,
K² and K³ each independently of one another represent a —CH₂—, —CHR^{8a}—, —CR^{8b}R^{8c}—, or a —C(O)— group, wherein R^{8a}/R^{8b}/R^{8c} each independently of one another represent a C₁₋₅-alkyl group which may be substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides a nitrogen atom may be replaced by a —C(O)— group, with the proviso that a heteroatom introduced by R^{8b}or R^{8c} may not be separated from X in formula I by only one carbon atom, and
   in total formula (II) should contain a maximum of four groups selected from among R^{7a}, R^{7b}, R^{7c}, R^{8a}, R^{8b}, and R^{8c},
X denotes a —N(R¹)— group, wherein R¹ denotes a hydrogen atom or a C₁₋₅-alkyl, C₃₋₅-alkenyl-CH₂—, C₃₋₅-alkynyl-CH₂—, C₃₋₆-cycloalkyl, C₄₋₆-cycloalkenyl group, wherein the methylene and methyl groups present in the groups mentioned previously may additionally be substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes a sulfur atom, a —C(R¹⁰)═N—, —N═C(R¹⁰)—, or —C(R¹⁰)═C(R¹¹)— group,
A² denotes either a nitrogen atom or a ═C(R¹²)— group, wherein R¹⁰, R¹¹, and R¹² each independently of one another represent a hydrogen, fluorine, chlorine, bromine atom, or a C₁₋₅-alkyl, —CF₃, cyano, carboxy, C₁₋₅-alkoxycarbonyl, hydroxy, C₁₋₃-alkoxy, CF₃O—, CHF₂O—, CH₂FO— group,
L denotes a substituted ring system of formula (IIa) 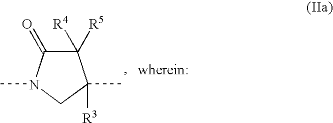
R³ denotes a hydrogen atom,
R⁴ and R⁵ each independently of one another represent a hydrogen atom, a hydroxy group, an —OR⁹ group, a C₂₋₆-alkenyl, or C₂₋₆-alkynyl group,
   a straight-chain or branched C₁₋₆-alkyl group, wherein the hydrogen atoms of the straight-chain or branched C₁₋₆-alkyl group may optionally be wholly or partly replaced by fluorine atoms, and wherein the straight-chain or branched C₁₋₆-alkyl group may optionally be substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c} group, and additionally a methylene group adjacent to an abovementioned —NR^{8c} group may be replaced by a carbonyl group,
   a phenyl or heteroaryl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a phenyl-C₁₋₅-alkyl or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which may optionally be substituted in the C₁₋₅-alkyl moiety by a hydroxy or a C₁₋₅-alkyloxy group, wherein the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group,
   a 3- to 7-membered cycloalkyl, cycloalkyleneimino, cycloalkyl-C₁₋₅-alkyl, or cycloalkyleneimino-C₁₋₃-alkyl group, wherein in 4- to 7-membered cyclic groups in the cyclic moiety a methylene group may optionally be replaced by a —N(R^{8c})group, an oxygen or sulfur atom, or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b}) or —N(R^{8b})C(O)N(R^{8b}) or —N(R^{8b})S(O)₂N(R^{8b}) group, with the proviso that a defined 3- to 7-membered cycloalkyl, cycloalkyleneimino, cycloalkyl-C₁₋₅-alkyl, or cycloalkyleneimino-C₁₋₃-alkyl group as hereinbefore wherein two heteroatoms selected from among oxygen and nitrogen are separated from one another by precisely one optionally substituted —CH₂— group, is excluded, wherein a 3- to 7-membered cycloalkyl, cycloalkyleneimino, cycloalkyl-C₁₋₅-alkyl, or cycloalkyleneimino-C₁₋₃-alkyl group as hereinbefore defined may be substituted at one or two —CH₂ groups by one or two C₁₋₃-alkyl groups in each case,
   with the proviso that R⁴ and R⁵ cannot simultaneously be defined as hydroxy or —OR⁹ groups, and wherein:
   R⁹ denotes a straight-chain or branched C₁₋₆-alkyl group, wherein the hydrogen atoms of the straight-chain or branched C₁₋₆-alkyl group may optionally be wholly or partly replaced by fluorine atoms, and wherein the straight-chain or branched C₁₋₆-alkyl group may optionally be substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-Cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylarnino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c}— group, and additionally a methylene group adjacent to an abovementioned —NR^{8c}— group may be replaced by a carbonyl group, with the proviso that the replacement of hydrogen atoms of the first carbon atom of the straight-chain or branched C₁₋₆-alkyl group by substituents selected from among oxygen, sulfur, or nitrogen is excluded,
   a phenyl, heteroaryl, phenyl-C₁₋₅-alkyl, or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a 3- to 7-membered cycloalkyl, cycloalkyl-C₁₋₅-alkyl, or cycloalkyleneimino-C₂₋₃-alkyl group, wherein in 4- to 7-membered cyclic groups in the cyclic moiety a methylene group may optionally be replaced by a —N(R^{8c})— group, an oxygen or sulfur atom, or a —S(O)— or —S(O)₂— group, or wherein in 4- to 7-membered cyclic groups in the cyclic moiety two adjacent methylene groups together may optionally be replaced by a —C(O)N(R^{8b})— or —S(O)₂N(R^{8b})— group, or wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyl-C₁₋₅-alkyl, or cycloalkyleneimino-C₂₋₃-alkyl group as hereinbefore defined wherein two heteroatoms selected from among oxygen and nitrogen are separated from one another by precisely one optionally substituted —CH₂— group, is excluded, wherein a 3- to 7-membered cycloalkyl, cycloalkyl-C₁₋₅-alkyl, or cycloalkyleneimino-C₂₋₃-alkyl group as hereinbefore defined may be substituted at one or two —CH₂— groups by one or two C₁₋₃-alkyl groups in each case,
M denotes a —CH₂—, —CHR³—, —CR³R³— group, or a bond,
W denotes an oxygen or sulfur atom,
B denotes a thiophene ring according to formula (III) 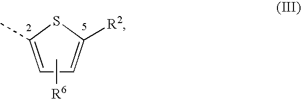
   which is bound to the carbonyl group in formula (I) via the 2-position and which is substituted in the 5-position by R² and optionally additionally by R⁶, wherein:
R² denotes a fluorine, chlorine, bromine, or iodine atom, or a methoxy, C₁₋₂-alkyl, or ethynyl group,
R⁶ denotes a hydrogen atom,
wherein, unless stated otherwise, by the term “heteroaryl group” mentioned hereinbefore in the definitions is meant a monocyclic 5- or 6-membered heteroaryl group, wherein:
   the 6-membered heteroaryl group contains one, two, or three nitrogen atoms, and
   the 5-membered heteroaryl group contains an imino group optionally substituted by a C₁₋₃-alkyl group, an oxygen or sulfur atom, or an imino group optionally substituted by a C₁₋₃-alkyl group, or an oxygen or sulfur atom and additionally a nitrogen atom, or an imino group optionally substituted by a C₁₋₃-alkyl group and two or three nitrogen atoms,
   and moreover a phenyl ring optionally substituted by a fluorine, chlorine, or bromine atom, a C₁₋₃-alkyl, hydroxy, C₁₋₃-alkyloxy group, amino, C₁₋₃-alkylamino, di-(C₁₋₃-alkyl)-amino, or C₃₋₆-cycloalkyleneimino group may be fused to the abovementioned monocyclic heteroaryl groups via two adjacent carbon atoms,
   and the bond is effected via a nitrogen atom or a carbon atom of the heterocyclic moiety or a fused-on phenyl ring,
wherein, unless stated otherwise, by the term “halogen atom” mentioned hereinbefore in the definitions is meant an atom selected from among fluorine, chlorine, bromine, and iodine,
wherein the alkyl, alkenyl, alkynyl, and alkoxy groups contained in the previously mentioned definitions which have more than two carbon atoms may, unless stated otherwise, be straight-chain or branched and the alkyl groups in the previously mentioned dialkylated groups, for example, the dialkylamino groups, may be identical or different,
and wherein the hydrogen atoms of the methyl or ethyl groups contained in the foregoing definitions, unless otherwise stated, may be wholly or partly replaced by fluorine atoms,
the tautomers, the enantiomers, the diastereomers, the mixtures, and the salts thereof

A fourth embodiment of the present invention encompasses those compounds of general formula (I), wherein:
D denotes a substituted bicyclic ring system of formula (II) 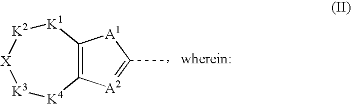
K¹ and K⁴ each independently of one another represent a —CH₂—, —CHR^{7a}—, —CR^{7b}R^{7c}—, or a —C(O)— group, wherein R^{7a}/R^{7b}/R^{7c} each independently of one another represent a fluorine atom, a hydroxy, C₁₋₅-alkoxy group, a C₁₋₅-alkyl group which may be substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂— group, or two groups R^{7b}/R^{7c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides an N atom may be replaced by a —C(O)— group,
K² and K³ each independently of one another represent a —CH₂—, CHR^{8a}—, —CR^{8b}R^{8c}—, or a —C(O)— group, wherein R^{8a}/R^{8b}/R^{8c} each independently of one another represent a C₁₋₅-alkyl group which may be substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom may form a 3-, 4-, 5-, 6-, or 7-membered saturated carbocycle or a cyclopentene, cyclohexene, oxetan, tetrahydrofuran, tetrahydropyran ring, wherein the methylene groups thereof may be substituted by one or two C₁₋₃-alkyl or —CF₃ groups, and/or the methylene groups thereof, if they are not bound to a heteroatom, may be substituted by one or two fluorine atoms, and/or wherein a —CH₂— group besides a nitrogen atom may be replaced by a —C(O)— group, with the proviso that a heteroatom introduced by R^{8b} or R^{8c} cannot be separated from X in formula I by only one carbon atom, and
   in total formula (II) should contain a maximum of four groups selected from among R^{7a}, R^{7b}, R^{7c}, R^{8a}, R^{8b}, and R^{8c},
X denotes a —N(R¹)— group, wherein R¹ denotes a hydrogen atom or a C₁₋₅-alkyl, C₃₋₅-alkenyl-CH₂, C₃₋₅-alkynyl-CH₂, C₃₋₆-cycloalkyl, C₄₋₆-cycloalkenyl group, wherein the methylene and methyl groups present in the groups mentioned previously may additionally be substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes a sulfur atom, a —C(R¹⁰)═N—, —N═C(R¹⁰)—, or —C(R¹⁰)═C(R¹¹)— group,
A² denotes either a nitrogen atom or a ═C(R¹²)— group, wherein R¹⁰, R¹¹, and R¹² each independently of one another represent a hydrogen, fluorine, chlorine, bromine atom, or a C₁₋₅-alkyl, —CF₃, cyano, carboxy, C₁₋₅-alkoxycarbonyl, hydroxy, C₁₋₃-alkoxy, CF₃O—, CHF₂O—, CH₂FO— group,
L denotes a substituted ring system of formula (IIa) 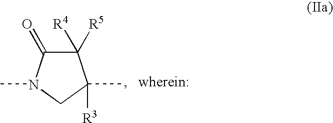
R³ denotes a hydrogen atom,
R⁴ denotes a hydrogen atom, a straight-chain or branched C₁₋₄-alkyl group, wherein the hydrogen atoms of the straight-chain or branched C₁₋₄-alkyl group may optionally be wholly or partly replaced by fluorine atoms, and which may optionally be substituted by a C₁₋₃-alkoxy group, wherein the hydrogen atoms of the C₁₋₃-alkoxy group may be wholly or partly replaced by fluorine atoms,
R⁵ denotes a hydrogen atom, a hydroxy group, an —OR⁹ group, a C₂₋₄-alkenyl, or C₂₋₄-alkynyl group,
   a straight-chain or branched C₁₋₄-alkyl group, wherein the hydrogen atoms of the straight-chain or branched C₁₋₄-alkyl group may optionally be wholly or partly replaced by fluorine atoms, and wherein the straight-chain or branched C₁₋₄-alkyl group may optionally be substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₄-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c} group, and additionally a methylene group adjacent to an abovementioned —NR^{8c} group may be replaced by a carbonyl group,
   a phenyl or heteroaryl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
   a phenyl-C₁₋₅-alkyl or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which may optionally be substituted in the C₁₋₅-alkyl moiety by a hydroxy or a C₁₋₅-alkyloxy group, wherein the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group, and wherein:
   R⁹ denotes a straight-chain or branched C₁₋₄-alkyl group, wherein the hydrogen atoms of the straight-chain or branched C₁₋₄-alkyl group may optionally be wholly or partly replaced by fluorine atoms, and wherein the straight-chain or branched C₁₋₄-alkyl group may optionally be substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxy-carbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group may optionally be wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group may be replaced by an oxygen or sulfur atom, by a carbonyl, sulfinyl, sulfonyl, or —NR^{8c}— group, and additionally a methylene group adjacent to an abovementioned —NR^{8c}— group may be replaced by a carbonyl group, with the proviso that the replacement of hydrogen atoms of the first carbon atom of the straight-chain or branched C₁₋₆-alkyl group by substituents selected from among oxygen, sulfur, or nitrogen is excluded,
   a phenyl, heteroaryl, phenyl-C₁₋₅-alkyl, or heteroaryl-C₁₋₅-alkyl group, which may optionally be mono- to tri-substituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
M denotes a —CH₂— group or a bond,
W denotes an oxygen atom,
B denotes a thiophene ring according to formula (III) 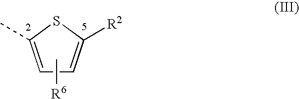
   which is bound to the carbonyl group in formula (I) via the 2-position and which is substituted in the 5-position by R² and optionally additionally by R⁶, where
R² denotes a fluorine, chlorine, bromine, or iodine atom, or a methoxy, C₁₋₂-alkyl, or ethynyl group,
R⁶ denotes a hydrogen atom,
wherein, unless stated otherwise, by the term “heteroaryl group” mentioned hereinbefore in the definitions is meant a monocyclic 5- or 6-membered heteroaryl group, wherein:
   the 6-membered heteroaryl group contains one, two, or three nitrogen atoms, and
   the 5-membered heteroaryl group contains an imino group optionally substituted by a C₁₋₃-alkyl group, an oxygen or sulfur atom, or an imino group optionally substituted by a C₁₋₃-alkyl group, or an oxygen or sulfur atom and additionally a nitrogen atom, or an imino group optionally substituted by a C₁₋₃-alkyl group and two or three nitrogen atoms,
   and moreover a phenyl ring optionally substituted by a fluorine, chlorine, or bromine atom, a C₁₋₃-alkyl, hydroxy, C₁₋₃-alkyloxy group, amino, C₁₋₃-alkylamino, di-(C₁₋₃-alkyl)-amino, or C₃₋₆-cycloalkyleneimino group may be fused to the abovementioned monocyclic heteroaryl groups via two adjacent carbon atoms,
   and the bond is effected via a nitrogen atom or a carbon atom of the heterocyclic moiety or a fused-on phenyl ring,
wherein, unless stated otherwise, by the term “halogen atom” mentioned hereinbefore in the definitions is meant an atom selected from among fluorine, chlorine, bromine, and iodine,
wherein the alkyl, alkenyl, alkynyl, and alkoxy groups contained in the previously mentioned definitions which have more than two carbon atoms may, unless stated otherwise, be straight-chain or branched, and the alkyl groups in the previously mentioned dialkylated groups, for example, the dialkylamino groups, may be identical or different,
and wherein the hydrogen atoms of the methyl or ethyl groups contained in the foregoing definitions, unless otherwise stated, may be wholly or partly replaced by fluorine atoms,
the tautomers, the enantiomers, the diastereomers, the mixtures, and the salts thereof.

A fifth embodiment of the present invention encompasses those compounds of general formula (I), wherein:
D denotes a substituted bicyclic ring system of formula (II) 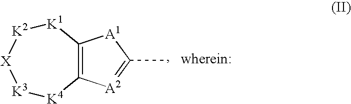
K¹ and K⁴ each independently of one another represent a —CH₂, —CHR^{7a}, —CR^{7b}R^{7c}, or a —C(O) group, wherein R^{7a}/R^{7b}/R^{7c} each independently of one another represent a C₁₋₂-alkyl group which may be substituted by one to three fluorine atoms;
K² and K³ each denote a —CH₂— group;
X denotes a —N(R¹)— group, wherein R¹ denotes a hydrogen atom or a C₁₋₅-alkyl or C₃₋₄-cyclo-alkyl group, wherein the methylene and methyl groups present in the groups mentioned previously may additionally be substituted by a hydroxy group, as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes a sulfur atom or —C(R¹⁰)═C(R¹¹)— group,
A² denotes either a nitrogen atom or a ═C(R¹²)— group, wherein R¹⁰, R¹¹, and R¹² each independently of one another represent a hydrogen, fluorine, or chlorine atom, or a C₁₋₅-alkyl, —CF₃, methoxy, CF₃O—, CHF₂O—, CH₂FO— group,
L denotes a substituted ring system of formula (IIa) 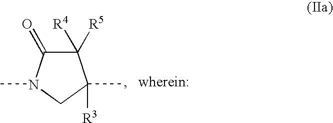
R³ denotes a hydrogen atom,
R⁴ denotes a hydrogen atom,
R⁵ denotes a hydrogen atom, a C₂₋₄-alkenyl, or C₂₋₄-alkynyl group,
   a straight-chain or branched C₁₋₄-alkyl group, wherein the hydrogen atoms of the straight-chain or branched C₁₋₄-alkyl group may optionally be wholly or partly replaced by fluorine atoms, and wherein the straight-chain or branched C₁₋₄-alkyl group may optionally be substituted by a hydroxy, C₁₋₅-alkyloxy group, or a di-(C₁₋₅-alkyl)-aminocarbonyl group, wherein the hydrogen atoms of the C₁₋₅-alkyloxy group may optionally be wholly or partly replaced by fluorine atoms,
   a phenyl-C₁₋₃-alkyl or heteroaryl-C₁₋₃-alkyl group,
M denotes a —CH₂— group or a bond,
W denotes an oxygen atom,
B denotes a thiophene ring according to formula (III) 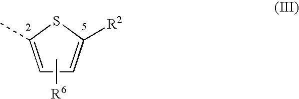
   which is bound to the carbonyl group in formula (I) via the 2-position and which is substituted in the 5-position by R² and optionally additionally by R⁶, wherein
R² denotes a chlorine or bromine atom, or an ethynyl group,
R⁶ denotes a hydrogen atom,
wherein, unless stated otherwise, by the term “heteroaryl group” mentioned hereinbefore in the definitions is meant a monocyclic 5- or 6-membered heteroaryl group, wherein:
   the 6-membered heteroaryl group contains one, two, or three nitrogen atoms, and
   the 5-membered heteroaryl group contains an imino group optionally substituted by a C₁₋₃-alkyl group, an oxygen or sulfur atom, or an imino group optionally substituted by a C₁₋₃-alkyl group, or an oxygen or sulfur atom and additionally a nitrogen atom, or an imino group optionally substituted by a C₁₋₃-alkyl group and two or three nitrogen atoms,
   and the bond is effected via a nitrogen atom or a carbon atom of the heterocyclic moiety or a fused-on phenyl ring,
wherein, unless stated otherwise, by the term “halogen atom” mentioned hereinbefore in the definitions is meant an atom selected from among fluorine, chlorine, bromine, and iodine,
wherein the alkyl, alkenyl, alkynyl, and alkoxy groups contained in the previously mentioned definitions which have more than two carbon atoms may, unless stated otherwise, be straight-chain or branched, and the alkyl groups in the previously mentioned dialkylated groups, for example, the dialkylamino groups, may be identical or different,
and wherein the hydrogen atoms of the methyl or ethyl groups contained in the foregoing definitions, unless otherwise stated, may be wholly or partly replaced by fluorine atoms,
the tautomers, the enantiomers, the diastereomers, the mixtures, and the salts thereof.

A sixth embodiment of the present invention encompasses those compounds of general formula (I), corresponding to embodiments 1, 2, 3, 4, or 5, wherein:
D denotes a substituted bicyclic ring system of formula (II) 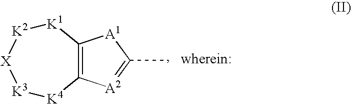
K¹ and K⁴ each independently of one another represent a —CH₂—, —CHR^{7a}—, —CR^{7b}R^{7c}—, or a —C(O)— group, wherein R^{7a}/R^{7b}/R^{7c} each independently of one another represent a C₁₋₂-alkyl group which may be substituted by one to three fluorine atoms,
K² and K³ each denote a —CH₂— group;
X denotes a —N(R¹)— group, wherein R¹ denotes a hydrogen atom or a C₁₋₅-alkyl or C₃₋₄-cycloalkyl group, wherein the methylene and methyl groups present in the groups mentioned previously may additionally be substituted by a hydroxy group, as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes a sulfur atom;
A² denotes a nitrogen atom.

A seventh embodiment of the present invention encompasses those compounds of general formula (I) corresponding to embodiments 1, 2, 3, 4, or 5, wherein:
D denotes a substituted bicyclic ring system of formula (II) 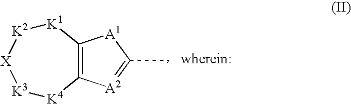
K¹ and K⁴ each independently of one another represent a —CH₂—, —CHR^{7a}—, —CR^{7b}R^{7c}, or a —C(O)— group, wherein R^{7a}/R^{7b}/R^{7c} each independently of one another represent a C₁₋₂-alkyl group which may be substituted by one to three fluorine atoms,
K² and K³ each denote a —CH₂— group;
X denotes a —N(R¹)— group, wherein R¹ denotes a hydrogen atom or a C₁₋₅-alkyl or C₃₋₄-cycloalkyl group, wherein the methylene and methyl groups present in the groups mentioned previously may additionally be substituted by a hydroxy group, as long as the methylene or methyl groups are not directly bound to a heteroatom selected from among O, N, or S, and/or one to three hydrogen atoms may be replaced by fluorine atoms, as long as the methylene or methyl groups are not directly bound to a heteroatom selected from among O, N, or S,
A¹ denotes a —C(R¹⁰)═C(R¹¹)— group,
A² denotes a ═C(R¹²)— group, wherein R¹⁰, R¹¹, and R¹² each independently of one another represent a hydrogen, fluorine, or chlorine atom, or a C₁₋₅-alkyl, —CF₃, methoxy, CF₃O—, CHF₂O—, CH₂FO— group.

An eighth embodiment of the present invention encompasses those compounds of general formula (I) corresponding to embodiments 1, 2, 3, 4, 5, 6, or 7, wherein M denotes a bond.

The following preferred compounds of general formula (I) are mentioned by way of example, both as the tautomers, the enantiomers, the diastereomers, the mixtures, and the salts thereof:
(1) (R)-5-bromothiophene-2-carboxylic acid-[1-(3-methyl-2,3,4,5-tetrahydro-1H-benzo[d]azepin-7-yl)-5-oxopyrrolidin-3-yl]amide
(2) (R)-5-chlorothiophene-2-carboxylic acid-[1-(3-methyl-2,3,4,5-tetrahydro-1H-benzo[d]azepin-7-yl)-5-oxopyrrolidin-3-yl]amide
(3) (R)-5-ethynyl-thiophene-2-carboxylic acid-[1-(3-methyl-2,3,4,5-tetrahydro-1H-benzo[d]azepin-7-yl)-5-oxopyrrolidin-3-yl]amide
(4) tert-butyl (R)-2-{4-[(5-bromothiophene-2-carbonyl)amino]-2-oxopyrrolidine-1-yl}-4,5,7,8-tetrahydrothiazolo[4,5-d]azepine-6-carboxylate
(5) (R)-5-chlorothiophene-2-carboxylic acid-[1-(3-methyl-2,3,4,5-tetrahydro-1H-benzo[d]azepin-7-yl)-5-oxopyrrolidin-3-yl]amide
(6) 5-chlorothiophene-2-carboxylic acid-[(3R,4R)-1-(3-methyl-2,3,4,5-tetrahydro-1H-benzo[d]azepin-7-yl)-5-oxo-4-propylpyrrolidin-3-yl]amide
(7) 5-bromothiophene-2-carboxylic acid-[(3R,4R)-1-(3-methyl-2,3,4,5-tetrahydro-1H-benzo[d]azepin-7-yl)-5-oxo-4-propylpyrrolidin-3-yl]amide
(8) 5-bromothiophene-2-carboxylic acid-[(3R,4R)-4-methoxymethyl-1-(3-methyl-2,3,4,5-tetrahydro-1H-benzo[d]azepin-7-yl)-5-oxo-4-propylpyrrolidin-3-yl]amide
(9) 5-chlorothiophene-2-carboxylic acid-[(3R,4R)-4-(2-methoxy-ethyl)-1-(3-methyl-2,3,4,5-tetrahydro-1H-benzo[d]azepin-7-yl)-5-oxopyrrolidin-3-yl]amide
(10) 5-chlorothiophene-2-carboxylic acid-[(3R,4R)-4-butyl-1-(3-methyl-2,3,4,5-tetrahydro-1H-benzo[d]azepin-7-yl)-5-oxopyrrolidin-3-yl]amide
(11) 5-bromothiophene-2-carboxylic acid-[(3R,4R)-4-butyl-1-(3-methyl-2,3,4,5-tetrahydro-1H-benzo[d]azepin-7-yl)-5-oxopyrrolidin-3-yl]amide
(12) 5-chlorothiophene-2-carboxylic acid-[(3R,4R)-4-ethyl-1-(3-methyl-2,3,4,5-tetrahydro-1H-benzo[d]azepin-7-yl)-5-oxopyrrolidin-3-yl]amide
(13) 5-bromothiophene-2-carboxylic acid-[(3R,4R)-4-ethyl-1-(3-methyl-2,3,4,5-tetrahydro-1H-benzo[d]azepin-7-yl)-5-oxopyrrolidin-3-yl]amide
(14) (R)-5-chlorothiophene-2-carboxylic acid-[5-oxo-1-(2,3,4,5-tetrahydro-1H-benzo[d]azepin-7-yl)pyrrolidine-7-yl]amide
(15) (R)-5-bromothiophene-2-carboxylic acid-[5-oxo-1-(5,6,7,8-tetrahydro-4H-thiazolo[4,5d]azepin-2-yl)pyrrolidin-3-yl]amide
(16) (R)-5-chlorothiophene-2-carboxylic acid-[1-(3-isopropyl-2,3,4,5-tetrahydro-1H-benzo[d]azepin-7-yl)-5-oxopyrrolidin-3-yl]amide
(17) (R)-5-bromothiophene-2-carboxylic acid-[1-(6-methyl-5,6,7,8-tetrahydro-4H-thiazolo[4,5-d]azepin-2-yl)-5-oxopyrrolidin-3-yl]amide
(18) (R)-5-chlorothiophene-2-carboxylic acid-[1-(3-ethyl-2,3,4,5-tetrahydro-1H-benzo[d]azepin-7-yl)-5-oxopyrrolidin-3-yl]amide
(19) 5-chlorothiophene-2-carboxylic acid-[(3R,4R)-1-(6-methyl-5,6,7,8-tetrahydro-4H-thiazolo[4,5-d]azepin-2-yl)-5-oxo-4-propylpyrrolidin-3-yl]amide
(20) 5-bromothiophene-2-carboxylic acid-[(3R,4R)-1-(6-methyl-5,6,7,8-tetrahydro-4H-thiazolo[4,5-d]azepin-2-yl)-5-oxo-4-propylpyrrolidin-3-yl]amide
(21) 5-ethynyl-thiophene-2-carboxylic acid-[(3R,4R)-1-(6-methyl-5,6,7,8-tetrahydro-4H-thiazolo[4,5-d]azepin-2-yl)-5-oxo-4-propylpyrrolidin-3-yl]amide
(22) 5-bromothiophene-2-carboxylic acid-[1-(3-methyl-2,3,4,5-tetrahydro-1H-benzo[d]azepin-7-yl)-5-oxopyrrolidin-3-ylmethyl]amide
(23) 5-chlorothiophen-2-thiocarboxylic acid-[1-(3-methyl-2,3,4,5-tetrahydro-1H-benzo[d]azepin-7-yl)-5-oxopyrrolidin-3-ylmethyl]amide
(24) 5-chlorothiophen-2-thiocarboxylic acid-[1-(1.1,3-trimethyl-2,3,4,5-tetrahydro-1H-benzo[d]azepin-7-yl)-5-oxopyrrolidin-3-ylmethyl]amide.

The invention also relates to physiologically acceptable salts of the compounds according to the previously defined embodiments and the Examples.

The invention also relates to pharmaceutical compositions containing a compound or a physiologically acceptable salt of a compound according to the previously defined embodiments and the Examples, optionally together with one or more inert carriers and/or diluents.

The invention also relates to the use of a compound or a physiologically acceptable salt of a compound according to the embodiments defined above and the Examples, for preparing a pharmaceutical composition with an inhibitory effect on factor Xa and/or an inhibitory effect on related serine proteases.

The invention also relates to a process for preparing a pharmaceutical composition, characterized in that by a non-chemical method a compound or a physiologically acceptable salt of a compound according to the embodiments defined above and the Examples is incorporated in one or more inert carriers and/or diluents.

According to the invention the compounds of general formula (I) are obtained by methods known per se, for example, by the following methods:

(a) The preparation of a compound of general formula (I) wherein A¹ and A², K¹ to K⁴, X, L, M, and R¹ to R⁶ are defined as in embodiment 1 and which may optionally be protected at any amino, hydroxy, carboxy, or thiol groups present by the usual protective groups such as, for example, those described in T. W. Greene and P. G. M. Wuts in “Protective Groups in Organic Synthesis” and the protective groups of which may be cleaved in a manner known from the literature, is described in the exemplifying embodiments or may be carried out, for example, according to one of the following formula Schemes 1 and 2.

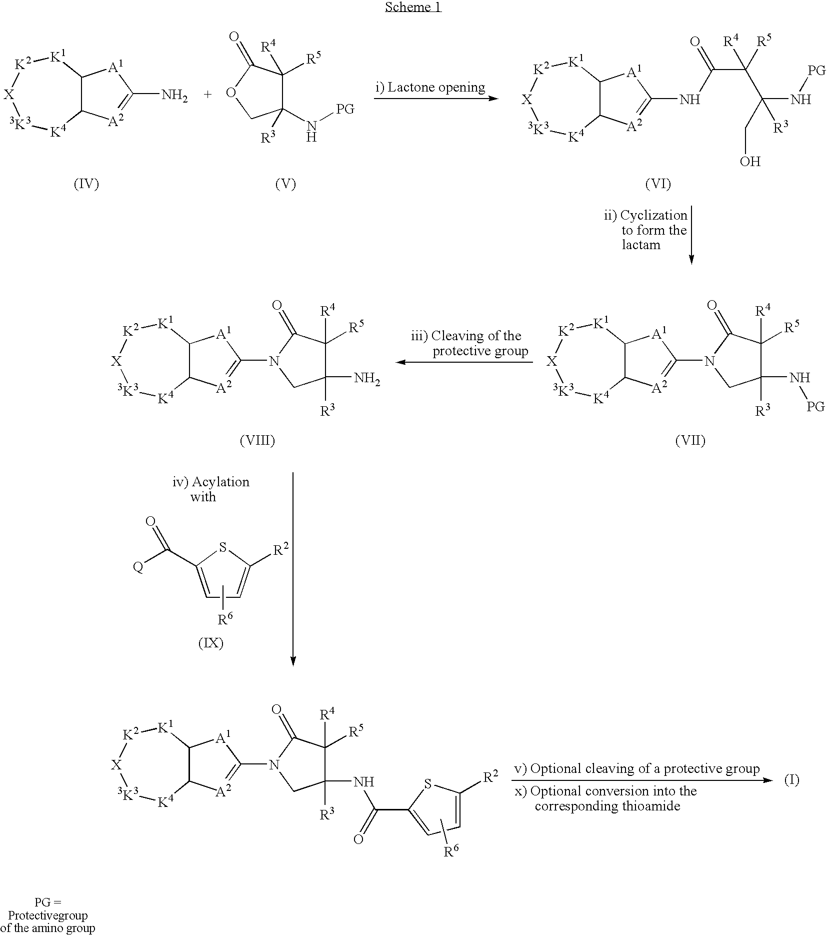

Alternatively compounds of general formula (Ia) may also be converted by analogous ring-opening of the lactone of general formula (Va) 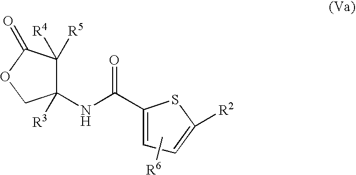
and subsequent cyclization into the corresponding pyrrolidinones (Ia).

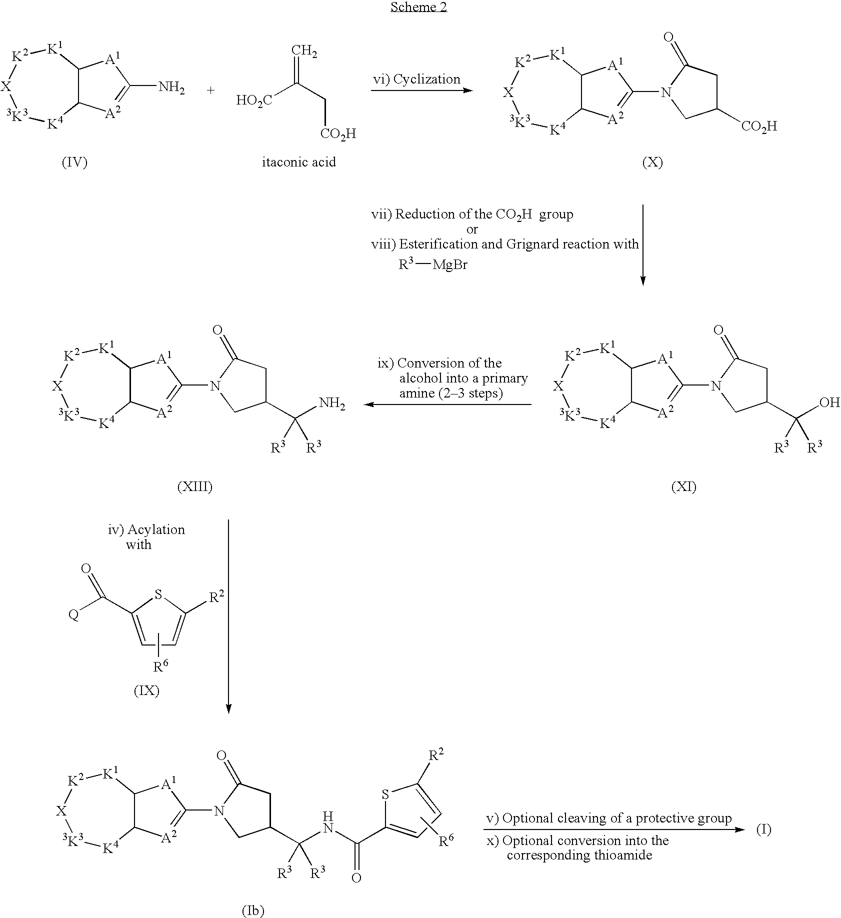

In Schemes 1 and 2, Q denotes a hydroxy or C₁₋₄-alkoxy group, a halogen atom or a alkoxycarbonyloxy or acyloxy group and PG denotes a protective group known from the literature for the amino function such as, for example, a tert-butoxycarbonyl, benzyloxycarbonyl, or a trifluoroacetyl group.

The reaction steps i) to ix) described in Schemes 1 and 2 may, for example, be carried out as described in the Examples or under conditions known from the literature, for example, as follows:

i) Ring-Opening of the Lactone (V) with the Amine (I)

The amine of general formula (IV) is activated with an organoaluminum compound such as, for example, trimethylaluminum, triethylaluminum, tripropylaluminum, triisobutylaluminum, tributylaluminum, or triphenylaluminum in a solvent or mixture of solvents such as dichloromethane, toluene, xylene, benzene, hexane, cyclohexane, heptane, or tetrahydrofuran, at a temperature of −100° C. to 100° C., but preferably between −80° C. and 80° C., and reacted with the lactone of general formula (V) or (Va).

ii) Cyclization to Form the Pyrrolidinone

The lactamization may be carried out under Mitsunubo conditions, expediently in an inert solvent or mixture of solvents such as, for example, tetrahydrofuran, dioxane, benzene, toluene, xylene, or acetonitrile, in the presence of phosphines such as, for example, triphenylphosphine or tributylphosphine, with dialkyl azodicarboxylates such as, for example, diethyl azodicarboxylate, diisopropyl azodicarboxylate, or di(tert-butyl)azodicarboxylate, for example, at a temperature of −50° C. to 200° C., but preferably between −20° C. and 150° C.

iii) or v) Cleaving a Protective Group in Scheme 1 and Scheme 2:

The optional subsequent cleaving of any protective group used is carried out hydrolytically, for example, in an aqueous solvent, e.g., in water, isopropanol/water, tetrahydrofuran/water, or dioxane/water, in the presence of an acid such as trifluoroacetic acid, hydrochloric acid, or sulfuric acid, or in the presence of an alkali metal base such as lithium hydroxide, sodium hydroxide, or potassium hydroxide, or by ether cleavage, e.g., in the presence of iodotrimethylsilane, at temperatures between 0° C. and 100° C., preferably at temperatures between 10° C. and 50° C.

A benzyl, methoxybenzyl, or benzyloxycarbonyl group may, however, be cleaved hydrogenolytically, e.g., with hydrogen in the presence of a catalyst such as palladium/charcoal in a solvent such as methanol, ethanol, ethyl acetate, dimethylformamide, dimethylformamide/acetone, or glacial acetic acid, optionally with the addition of an acid such as hydrochloric acid at temperatures between 0° C. and 50° C., but preferably at room temperature, and under a hydrogen pressure of 1 to 7 bar, but preferably 1 to 5 bar.

A protective group may however also be cleaved by the methods described in T. W. Greene and P. G. M. Wuts in “Protective Groups in Organic Synthesis”.

iv) Acylation of an Amine (VIII) or (XII) with an Optionally Activated Carboxylic Acid (V)

The acylation is conveniently carried out with a corresponding halide or anhydride in a solvent such as methylene chloride, chloroform, carbon tetrachloride, ether, tetrahydrofuran, dioxane, benzene, toluene, acetonitrile, dimethylformamide, sodium hydroxide solution, or sulfolane, optionally in the presence of an inorganic or organic base at temperatures between −20° C. and 200° C., but preferably at temperatures between −10° C. and 160° C.

The acylation may, however, also be carried out with the free acid, optionally in the presence of an acid-activating agent or a dehydrating agent, for example, in the presence of isobutyl chloroformate, thionyl chloride, trimethylchlorosilane, hydrogen chloride, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, 1-hydroxybenzotriazole, N,N′-carbonyldiimidazole, N,N′-carbonylditriazole, O-(benzotriazol-1-yl)-N,N′,N′-tetramethyluroniumtetrafluoroborate/N-methylmorpholine, O-(benzotriazol-1-yl)-N,N,′,N′-tetramethyluroniumtetrafluoroborate/N-ethyldiisopropylamine, O-pentafluorophenyl-N,N,N′,N′-tetramethyluroniumhexafluorophosphate/triethylamine, N,N′-thionyldiimidazole, or triphenylphosphine/carbon tetrachloride, at temperatures between −20° C. and 200° C., but preferably at temperatures between −10° C. and 160° C.

vi) Tandem Michael Addition/Lactamization with Itaconic Acid

The tandem Michael addition/lactamization is conveniently carried out with itaconic acid at a temperature of 50° C.-250° C., but preferably at 80° C.-200° C., in the presence or absence of a solvent or mixture of solvents such as water, ethanol, propanol, butanol, toluene, xylene, chlorobenzene, tetralin, or diphenyl ether.

viii) Esterification and Grignard Reaction

Optional subsequent substitution with R³ groups is prepared by blocking the carboxylic acid function by esterification using methods known from the literature and reaction with Grignard compounds of the type R³—Mg—Br or R³—Mg—Cl in an inert solvent such as, for example, diethyl ether or tetrahydrofuran, at temperatures of −100° C. to +100° C.; but preferably between −80° C. and +80° C. Tertiary alcohols of general formula (XI) are thus formed.

vii) Reduction of the Carboxylic Acid to the Primary Alcohol of General Formula (XI)

The reduction of the carboxylic acid function may be carried out by methods known from the literature, by esterification or other activation methods (e.g., by conversion into an active ester or carbonyl chloride) and subsequent reduction with a borohydride such as, for example, sodium or lithium borohydride in a solvent or mixture of solvents such as, for example, methanol, water, tetrahydrofuran, or diethyl ether at temperatures between −100° C. and +100° C., but preferably between −80° C. and +100° C.

ix) Conversion of the Hydroxyl Compound of General Formula (XI) into a Primarey Amine

The conversion of the alcohol function into an amine is carried out in a two-step process by activation according to Mitsunobu analogously to ii). By reacting with phthalimide and subsequently liberating the amine with hydrazine or methylamine the amine of general formula (XII) is obtained.

Alternatively the hydroxyl function may also be converted into a leaving group such as, for example, mesylate, tosylate, iodide, or the like, by methods known from the literature. By subsequent nucleophilic substitution with a compound selected from among lithium, sodium, potassium azide, sodium, potassium phthalimide, 4-methoxybenzylamine, benzylamine, 2,4-dimethoxybenzylamine, dibenzylamine, potassium, or sodium cyanide, for example, and subsequent reduction by standard methods of the nitrogen-containing group thus introduced the amine of general formula (XII) is obtained.

Other methods of amide coupling are described, for example, in P. D. Bailey, I. D. Collier, K. M. Morgan in “Comprehensive Functional Group Interconversions”, Vol. 5, page 257ff., Pergamon, 1995, or in Houben-Weyl, Supplementary Volume 22, Thieme Verlag, 2003, and the literature cited therein, all of which are hereby incorporated by reference herein.

x) Reaction with Lawesson's Reajent to Obtain the Corresponding Thioamide

The conversion of the carbonyl group may be carried out using methods known from the literature, by reaction, for example, with Lawesson's reagent in an inert solvent or mixture of solvents such as, for example, toluene, benzene, or chlorobenzene at temperatures between −100° C. and +100° C., but preferably between −80° C. and +100° C.

(b) The Components of General Formula 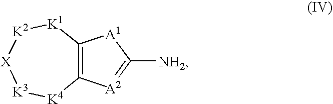
wherein A¹, A², K¹, K², K³, K⁴, and X are defined as mentioned in embodiment 1, and which may optionally be protected at any amino, hydroxy, carboxy, or thiol groups present by the usual protective groups such as, for example, those described in T. W. Greene and P. G. M. Wuts in “Protective Groups in Organic Synthesis” and the protective groups of which may be cleaved in a manner known from the literature in the course of the synthesis sequence to form compounds of formula (I), are known from the literature, or their synthesis is described in the exemplifying embodiments, or they may be prepared, for example, using methods of synthesis known from the literature or analogously to methods of synthesis known from the literature as described, for example, in DE4429079, U.S. Pat. No. 4,490,369, DE3515864, U.S. Pat. No. 5,175,157, DE1921861, WO85/00808 or in G. Bobowski et al., J. Heterocyclic Chem. 16, 1525, 1979, or in P. D. Johnson et al., Bioorg. Med. Chem. Lett. 2003, 4197.

For example, a compound of general formula (IV), wherein A¹, A², K¹, K², K³, K⁴, and X are defined as mentioned in embodiment 1, may be prepared by reduction of the nitro group of a compound of general formula (XII) 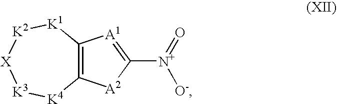
wherein A¹, A², K¹, K², K³, K⁴, and X are defined as mentioned in embodiment 1.

The reduction of the nitro group is, for example, conveniently carried out in a solvent or mixture of solvents such as water, aqueous ammonium chloride solution, hydrochloric acid, sulfuric acid, phosphoric acid, formic acid, acetic acid, acetic anhydride with base metals such as iron, zinc, tin, or sulfur compounds such as ammonium sulfide, sodium sulfide, or sodium dithionite, or by catalytic hydrogenation with hydrogen, for example, under a pressure between 0.5 and 100 bar, but preferably between 1 and 50 bar, or with hydrazine as reducing agent, conveniently in the presence of a catalyst such as, for example, Raney nickel, palladium charcoal, platinum oxide, platinum on mineral fibers, or rhodium, or with complex hydrides such as lithium aluminum hydride, sodium borohydride, sodium cyanoborohydride, diisobutylaluminum hydride, conveniently in a solvent or mixture of solvents such as water, methanol, ethanol, isopropanol, pentane, hexane, cyclohexane, heptane, benzene, toluene, xylene, ethyl acetate, methylpropionate, glycol, glycol dimethyl ether, diethylene glycol dimethyl ether, dioxane, tetrahydrofuran, N-methylpyrrolidinone, or N-ethyldiisopropylamine, N—C₁₋₅-alkylmorpholine, N—C₁₋₅-alkylpiperidine, N—C₁₋₅-alkylpyrrolidine, triethylamine, or pyridine, for example, at temperatures between −30° C. and 250° C., but preferably between 0° C. and 150° C.
(c) The Components of General Formula 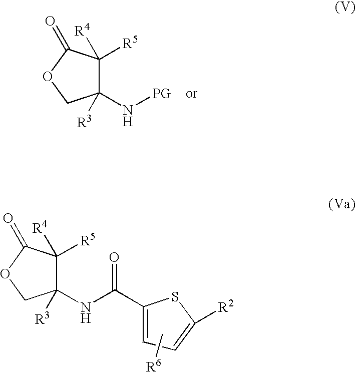
wherein R²-R⁶ are defined as mentioned in embodiment 1, and wherein PG denotes a protective group for the amino group, and which may optionally be protected at any amino, hydroxy, carboxy, or thiol groups present by the usual protective groups such as, for example, those described in T. W. Greene and P. G. M. Wuts in “Protective Groups in Organic Synthesis” and the protective groups of which may be cleaved in a manner known from the literature in the course of the synthesis sequence to form compounds of formula (I), are known from the literature, or their synthesis is described in the exemplifying embodiments, or they may be prepared, for example, using methods of synthesis known from the literature or by the following methods:
1) Reduction and Subsequent Lactonization of a Compound of General Formula 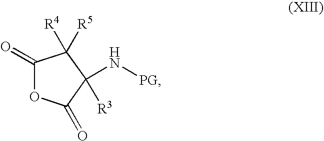
wherein PG denotes a protective group of the amino function, which may subsequently be cleaved by methods known from the literature, and R³ to R⁵ are defined as in the first embodiment.

The reduction to the intermediate hydroxy acid is, for example, conveniently carried out in a solvent or mixture of solvents such as tetrahydrofuran, dioxane, glycol dimethyl ether, diethylene glycol dimethyl ether, pentane, hexane, cyclohexane, heptane, benzene, toluene, or xylene with complex hydrides such as sodium borohydride, lithium borohydride, or sodium cyanoborohydride, for example, at temperatures between −80° C. and 250° C., but preferably between −30° C. and 150° C.

The subsequent lactonization of the intermediate is conveniently carried out, for example, in a solvent or mixture of solvents such as benzene, chlorobenzene, toluene, xylene, dichloromethane, chloroform, tetrachloromethane, 1,2-dichloroethane, in the presence of a catalyst such as p-toluenesulfonic acid, camphorsulfonic acid, or acid ion exchanger, optionally in the presence of a desiccant such as sodium sulfate, magnesium sulfate, or molecular sieves, for example, at temperatures between −30° C. and 250° C., but preferably between temperatures of 0° C. and 200° C. For example, this reaction may be carried out as described by G. J. McGarvey, J. M. Williams, R. N. Hiner, Y. Matsubara, and T. Oh, J. Am. Chem. Soc. 1986, 108, 4943-4952.

2) (Sequential) Alkylation of a Compound of General Formula 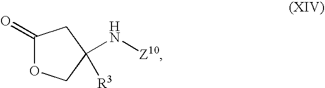
where R³ is defined as in the first embodiment and Z¹⁰ denotes a protective group of the amino function, which may subsequently be cleaved by methods known from the literature, but may also represent an acyl group of formula 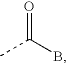
wherein B is defined as in the first embodiment, with a compound of general formula T-Z¹¹ (XV), wherein the group T denotes the groups R⁴ or R⁵ defined in the first embodiment, with the proviso that T cannot represent the group OR⁹, and Z¹¹ denotes a nucleofugic group, for example, an iodine, bromine, or chlorine atom, or a tosylate, triflate, or mesylate group.

The alkylation may be repeated with an identical or different alkylating agent of formula (XV), so as to obtain a,a-disubstituted lactones of compound (V) or (Va). The alkylations may be carried out analogously to A. El Hadri, A. Ahbouabdellah, U. Thomet, R. Baur, R. Furtmüller, E. Sigel, W. Sieghart, and R. H. Dodd, J. Med. Chem. 2002, 45, 2824-2831.

In the reactions described hereinbefore any reactive groups present such as hydroxy, carboxy, amino, alkylamino, or imino groups may be protected during the reaction by conventional protective groups which are cleaved again after the reaction.

For example a protecting group for a hydroxy group might be the methoxy, benzyloxy, trimethylsilyl, acetyl, benzoyl, tert-butyl, trityl, benzyl, or tetrahydropyranyl group.

Protecting groups for a carboxyl group might be the trimethylsilyl, methyl, ethyl, tert-butyl, benzyl, or tetrahydropyranyl group.

A protecting group for an amino, alkylamino, or imino group might be the acetyl, trifluoroacetyl, benzoyl, ethoxycarbonyl, tert-butoxycarbonyl, benzyloxycarbonyl, benzyl, methoxybenzyl, or 2,4-dimethoxybenzyl group and additionally, for the amino group, the phthalyl group.

For example, a protecting group for an ethynyl group might be the trimethylsilyl, diphenylmethylsilyl, tert-butyldimethylsilyl, or a 1-hydroxy-1-methylethyl group.

Other protective groups which may be used and their removal are described in T. W. Greene and P. G. M. Wuts, “Protective Groups in Organic Synthesis”, Wiley, 1991 and 1999, which is hereby incorporated by reference.

Any protective group used is optionally subsequently cleaved, for example, by hydrolysis in an aqueous solvent, e.g., in water, isopropanol/water, tetrahydrofuran/water, or dioxane/water, in the presence of an acid such as trifluoroacetic acid, hydrochloric acid, or sulfuric acid, or in the presence of an alkali metal base such as lithium hydroxide, sodium hydroxide, or potassium hydroxide, or by means of ether splitting, e.g., in the presence of iodotrimethylsilane, at temperatures between 0° C. and 100° C., preferably at temperatures between 10° C. and 50° C.

A benzyl, methoxybenzyl, or benzyloxycarbonyl group, however, is cleaved by hydrogenolysis, for example, with hydrogen in the presence of a catalyst such as palladium/charcoal in a solvent such as methanol, ethanol, ethyl acetate, dimethylformamide, dimethylformamide/acetone, or glacial acetic acid, optionally with the addition of an acid such as hydrochloric acid at temperatures between 0° C. and 50° C., but preferably at room temperature, and under a hydrogen pressure of 1 to 7 bar, but preferably 1 to 5 bar.

A methoxybenzyl group may also be cleaved in the presence of an oxidizing agent such as cerium (IV) ammonium nitrate in a solvent such as methylene chloride, acetonitrile, or acetonitrile/water at temperatures between 0° C. and 50° C., but preferably at room temperature.

A methoxy group is conveniently cleaved in the presence of boron tribromide in a solvent such as methylene chloride at temperatures between −35° C. and −25° C.

A 2,4-dimethoxybenzyl group, however, is preferably cleaved in trifluoroacetic acid in the presence of anisole.

A tert-butyl or tert-butyloxycarbonyl group is preferably cleaved by treatment with an acid such as trifluoroacetic acid or hydrochloric acid, optionally using a solvent such as methylene chloride, dioxane, or ether.

A phthalyl group is preferably cleaved in the presence of hydrazine or a primary amine such as methylamine, ethylamine, or n-butylamine in a solvent such as methanol, ethanol, isopropanol, toluene/water, or dioxane at temperatures between 20° C. and 50° C.

An allyloxycarbonyl group is cleaved by treatment with a catalytic amount of tetrakis-(triphenylphosphine)palladium (0), preferably in a solvent such as tetrahydrofuran and preferably in the presence of an excess of a base such as morpholine or 1,3-dimedone at temperatures between 0° C. and 100° C., preferably at room temperature and under inert gas, or by treatment with a catalytic amount of tris-(triphenylphosphine)rhodium (I) chloride in a solvent such as aqueous ethanol and optionally in the presence of a base such as 1,4-diazabicyclo[2,2,2]octane at temperatures between 20° C. and 70° C.

Moreover, the compounds of general formula I obtained may be resolved into their enantiomers and/or diastereomers.

Thus, for example, the compounds of general formula I obtained which occur as racemates may be separated by methods known per se (cf N. L. Allinger and E. L. Eliel in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.

The enantiomers are preferably separated by column separation on chiral phases or by recrystallization from an optically active solvent or by reacting with an optically active substance which forms salts or derivatives such as, e.g., esters or amides with the racemic compound, particularly acids and the activated derivatives or alcohols thereof, and separating the diastereomeric mixture of salts or derivatives thus obtained, e.g., on the basis of their differences in solubility, whilst the free antipodes may be released from the pure diastereomeric salts or derivatives by the action of suitable agents. Optically active acids in common use are, e.g., the D- and L-forms of tartaric acid or dibenzoyltartaric acid, di-o-tolyltartaric acid, malic acid, mandelic acid, camphorsulfonic acid, glutamic acid, aspartic acid, or quinic acid. An optically active alcohol may be, for example, (+) or (−)-menthol and an optically active acyl group in amides, for example, may be a (+)- or (−)-menthyloxycarbonyl.

Furthermore, the compounds of formula (I) obtained may be converted into the salts thereof, particularly for pharmaceutical use into the physiologically acceptable salts with inorganic or organic acids. Acids which may be used for this purpose include, for example, hydrochloric acid, hydrobromic acid, sulfuric acid, methanesulfonic acid, phosphoric acid, fumaric acid, succinic acid, lactic acid, citric acid, tartaric acid, or maleic acid.

Moreover, if the new compounds of formula (I) contain a carboxy group, they may subsequently, if desired, be converted into the salts thereof with inorganic or organic bases, particularly for pharmaceutical use into the physiologically acceptable salts thereof. Suitable bases for this purpose include, for example, sodium hydroxide, potassium hydroxide, cyclohexylamine, ethanolamine, diethanolamine, and triethanolamine.

As already mentioned hereinbefore, the compounds of general formula (I), and the tautomers, enantiomers, diastereomers, and physiologically acceptable salts thereof have valuable pharmacological properties, particularly an antithrombotic activity which is preferably based on an effect on thrombin or factor Xa, for example, on a thrombin-inhibiting or factor Xa-inhibiting activity, on a prolonging effect on the aPTT time and on an inhibitory effect on related sermne proteases such as, e.g., urokinase, factor VIIa, factor IX, factor XI, and factor

The compounds listed in the Experimental Section were investigated for their effect on the inhibition of factor Xa as follows:

Method:

Enzyme-kinetic measurement with chromogenic substrate. The quantity of p-nitroaniline (pNA) released from the colorless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Materials:

Tris(hydroxymethyl)aminomethane buffer (100 mmol) and sodium chloride (150 mmol), pH 8.0 plus 1 mg/mL Human Albumin Fraction V, protease-free

Factor Xa (Calbiochem), spec. activity: 217 IU/mg, final concentration: 7 IU/mL for each reaction mixture

Substrate S 2765 (Chromogenix), final concentration: 0.3 mmol/L (1 KM) for each reaction mixture

Test substance: final concentration 100, 30, 10, 3, 1, 0.3, 0.1, 0.03, 0.01, 0.003, 0.001 μmol/L.

Procedure:

10 μL of a 23.5-times concentrated starting solution of the test substance or solvent (control), 175 μL of TRIS/HSA buffer, and 25 μL of a 65.8 U/L Factor Xa working solution are incubated for 10 minutes at 37° C. After the addition of 25 μL of S 2765 working solution (2.82 mmol/L), the sample is measured in a photometer (SpectraMax 250) at 405 nm for 600 seconds at 37° C.

Evaluation:

1. Determining the maximum increase (deltaOD/minutes) over 21 measuring points.

2. Determining the % inhibition based on the solvent control.

3. Plotting a dosage/activity curve (% inhibition vs. substance concentration).

4. Determining the IC₅₀ by interpolating the X-value (substance concentration) of the dosage/activity curve at Y=50% inhibition.

All the compounds tested had an IC₅₀ value of less than 100 μmol/L.

The compounds prepared according to the invention are generally well tolerated.

In view of their pharmacological properties the new compounds and the physiologically acceptable salts thereof are suitable for the prevention and treatment of venous and arterial thrombotic diseases, such as, for example, the prevention and treatment of deep leg vein thrombosis, for preventing reocclusions after bypass operations or angioplasty (PT(C)A), and occlusion in peripheral arterial diseases, and for preventing and treating pulmonary embolism, disseminated intravascular coagulation and severe sepsis, for preventing and treating DVT in patients with exacerbated COPD, for treating ulcerative colitis, for preventing and treating coronary thrombosis, and for preventing stroke and the occlusion of shunts.

In addition, the compounds according to the invention are suitable for antithrombotic support in thrombolytic treatment, such as, for example, with alteplase, reteplase, tenecteplase, staphylokinase, or streptokinase, for preventing long-term restenosis after PT(C)A, for the prevention and treatment of ischemic events in patients with all forms of coronary heart disease, for preventing metastasis and the growth of tumors and inflammatory processes, e.g., in the treatment of pulmonary fibrosis, for preventing and treating rheumatoid arthritis, for preventing and treating fibrin-dependent tissue adhesions and/or the formation of scar tissue, and for promoting wound healing processes.

In view of their pharmacological properties the new compounds and the physiologically acceptable salts thereof are also suitable for the treatment of Alzheimer's and Parkinson's disease. One explanation for this arises, for example, from the following findings, from which it can be concluded that thrombin inhibitors or factor Xa inhibitors, by inhibiting thrombin formation or thrombin activity, may be valuable drugs for treating Alzheimer's and Parkinson's disease. Clinical and experimental studies indicate that neurotoxic mechanisms, for example, the inflammation which is associated with the activation of proteases of the clotting cascade, are involved in the dying of neurons following brain injury. Various studies point to the involvement of thrombin in neurodegenerative processes, for example, following a stroke, repeated bypass operations, or traumatic brain injury. An increased thrombin activity has been demonstrated some days after peripheral nerve damage, for example. It has also been shown that thrombin causes a neurite retraction, as well as glia proliferation, and apoptosis in primary cultures of neurons and neuroblastoma cells (for a summary see Neurobiol. Aging 2004, 25(6), 783-793). Moreover, various in vitro studies on the brains of patients with Alzheimer's disease indicated that thrombin plays a role in the pathogenesis of this disease (Neurosci. Lett. 1992, 146, 152-54). A concentration of immune-reactive thrombin has been detected in neurite plaques in the brains of Alzheimer's patients. It has been demonstrated in vitro that thrombin also plays a part in the regulation and stimulation of the production of the “Amyloid Precursor Protein” (APP) as well as in the cleaving of the APP into fragments which can be detected in the brains of Alzheimer's patients. Moreover, it has been demonstrated that the thrombin-induced microglial activation leads in vivo to the degeneration of nigral dopaminergic neurons. These findings lead one to conclude that microglial activation, triggered by endogenous substance(s) such as thrombin, for example, are involved in the neuropathological process of the cell death of dopaminergic neurons of the kind which occurs in patients with Parkinson's disease (J. Neurosci. 2003, 23, 5877-86).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day.

For this purpose, the compounds of formula (I) prepared according to the invention may be formulated, optionally together with other active substances, with one or more inert conventional carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose, or fatty substances such as hard fat or suitable mixtures thereof, to produce conventional galenic preparations such as plain or coated tablets, capsules, powders, suspensions, or suppositories.

The new compounds and the physiologically acceptable salts thereof may be used therapeutically in conjunction with acetylsalicylic acid, with inhibitors of platelet aggregation such as fibrinogen receptor antagonists (e.g., abciximab, eptifibatide, tirofiban, roxifiban), with physiological activators and inhibitors of the clotting system and the recombinant analogues thereof (e.g., Protein C, TFPI, antithrombin), with inhibitors of ADP-induced aggregation (e.g., clopidogrel, ticlopidine), with P₂T receptor antagonists (e.g., cangrelor), or with combined thromboxane receptor antagonists/synthetase inhibitors (e.g., terbogrel).

Experimental Section

The Examples that follow are intended to illustrate the invention, without restricting its scope.

As a rule, melting points, IR, UV, ¹H-NMR, and/or mass spectra have been obtained for the compounds prepared. Unless otherwise stated, R_{f} values were determined using ready-made silica gel 60 F₂₅₄ TLC plates (E. Merck, Darmstadt, Item No. 1.0571-4) without chamber saturation. The R_{f} values given under the heading Alox were determined using ready-made aluminum oxide 60 F₂₅₄ TLC plates (E. Merck, Darmstadt, Item No. 1.05713) without chamber saturation. The R_{f} values given under the heading Reversed-phase-8 (RP-8) were determined using ready-made RP-8 F₂₅₄ₛ TLC plates (E. Merck, Darmstadt, Item No. 1.15684) without chamber saturation. The ratios given for the eluants refer to units by volume of the solvents in question. For chromatographic purification silica gel made by Messrs Millipore (MATREX™, 35-70 μm) was used. Unless more detailed information is provided as to the configuration, it is not clear whether the products are pure stereoisomers or mixtures of enantiomers and diastereomers.

The following abbreviations are used in the test descriptions:

Boc tert-butoxycarbonyl

DCC N,N′-dicyclohexylcarbodiimide

DIPEA N-ethyl-diisopropylamine

DMSO dimethylsulfoxide

DMF N,N-dimethylformamide

DPPA diphenylphosphorylazide

NMM N-methyl-morpholine

NMP N-methylpyrrolidin-2-one

o ortho

PfTU O-pentafluorophenyl-N,N,N′,N′-tetramethyluronium-hexafluorophosphate

PPA propanephosphonic acidcycloanhydride

quant. quantitative

R_{f} retention factor

Rₜ retention time

RT or ambient temperature normal room temperature

rac. racemic

TBTU O-(benzotriazol-1-yl)-N,N,N′,N′-tetramethyluronium tetrafluoroborate

TEA triethylamine

TFA trifluoroacetic acid

THF tetrahydrofuran

Σ yield over all the steps described, carried out analogously

The HPLC data for Examples 3, 20, and 23 were generated under the following conditions:

Waters ZMD, Alliance 2695 HPLC, Waters 2700 Autosampler, Waters 996 diode array detector

The mobile phase used was:

A: water with 0.13% TFA

B: acetonitrile with 0.10% TFA

| time in min | % A | % B | flow rate in mL/min |
|---|---|---|---|
| 0.0 | 95 | 5 | 1.00 |
| 0.7 | 95 | 5 | 1.00 |
| 5.2 | 2 | 98 | 1.00 |
| 5.7 | 2 | 98 | 1.00 |
| 6.0 | 95 | 5 | 1.00 |
| 6.5 | 95 | 5 | 1.00 |

The stationary phase used was a Varian column, Microsorb 100 C₁₈ 3 μm, 4.6 mm×50 mm, batch No. 2231108 (column temperature: constant at 25° C.).

The diode array detection was carried out in the wavelength range 210-300 nm.

The HPLC data for all the other Examples were obtained under the following conditions:

Waters ZMD, Alliance 2695 HPLC, Waters 2700 Autosampler, Waters 2996 diode array detector

The mobile phase used was:

A: water with 0.10% TFA

B: acetonitrile with 0.10% TFA

| time in min | % A | % B | flow rate in mL/min |
|---|---|---|---|
| 0.0 | 95 | 5 | 1.00 |
| 0.1 | 95 | 5 | 1.00 |
| 3.1 | 2 | 98 | 1.00 |
| 4.5 | 2 | 98 | 1.00 |
| 5.0 | 95 | 5 | 1.00 |

The stationary phase used was an XTerra® column, MS C₁₈ 2.5 μm, 4.6 mm×30 mm (column temperature: constant at 25° C.).

The diode array detection was carried out in the wavelength range 210-300 nm.

### EXAMPLE 1

### (R)-5-bromothiophene-2-carboxylic acid-[1-(3-methyl-2,3,4,5-tetrahydro-1H-benzo[d]azepin-7-yl)-5-oxopyrrolidin-3-yl]amide (monotrifluoroacetate salt)

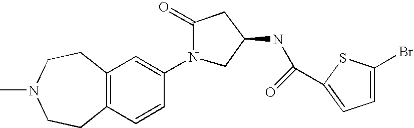

### (a) benzyl (R)-{2-hydroxy-1-[(3-methyl-2,3,4,5-tetrahydro-1H-benzo[d]azepin-7-ylcarbamoyl)methyl]ethyl}carbamate

2.00 g (7.74 mmol) of 7-amino-3-trifluoroacetyl-2,3,4,5-tetrahydro-1H-benzo[d]azepine is dissolved in 8 mL of dichloromethane and at ambient temperature combined with 4.1 mL (8.2 mmol) trimethylaluminum solution (2M in toluene). After 15 minutes, 1.82 g (7.74 mmol) of (R)-(5-oxotetrahydrofuran-3-yl)carbamate benzyl is added and the mixture is stirred for 16 hours at ambient temperature. Then it is acidified with 2N hydrochloric acid, diluted with water, and extracted three times with ethyl acetate. The combined organic phases are dried on sodium sulfate and evaporated to dryness. The crude product thus obtained is purified by chromatography on silica gel (eluant: dichloromethane/methanol 9:1). A white solid is obtained. Yield: 1.44 g (38%); R_{f} value: 0.27 (silica gel; dichloromethane/methanol=95:5); C₂₄H₂₆F₃N₃O₅ (493.48); mass spectrum: (M+H)⁺=494.

### (b) benzyl (R)-{5-oxo-1-[3-(2,2,2-trifluoroacetyl)-2,3,4,5-tetrahydro-1H-benzo[d]azepin-7-yl]pyrrolidin-3-yl}carbamate

1.44 g (2.92 mmol) of benzyl (R)-{2-hydroxy-1-[(3-methyl-2,3,4,5-tetrahydro-1H-benzo[d]azepin-7-ylcarbamoyl)methyl]ethyl}carbamate is dissolved in 4 mL of THF. While cooling with ice, a mixture of 740 mg (3.2 mmol) of di-tert-butylazodicarboxylate and 800 μL (3.2 mmol) of tributylphosphine in 3 mL of THF is added. The mixture is slowly heated to ambient temperature and stirred for 16 hours. Then the mixture is evaporated to dryness. The residue is purified by reversed-phase chromatography. Yield: 545 mg (39%); Rₜ value: 3.21 min; C₂₄H₂₄F₃N₃O₄ (475.46); mass spectrum: (M+H)⁺=476.

### (c) benzyl (R)-[5-oxo-1-(2,3,4,5-tetrahydro-1H-benzo[d]azepin-7-yl)pyrrolidin-3-yl]carbamate

500 mg (1.1 mmol) of benzyl (R)-{5-oxo-1-[3-(2,2,2-trifluoroacetyl)-2,3,4,5-tetrahydro-1H-benzo[d]azepin-7-yl]pyrrolidin-3-yl}carbamate is dissolved in a mixture of 10 mL of methanol and 5 mL of water, combined with 620 mg (4.5 mmol) of potassium carbonate, and stirred for 4.5 hours at ambient temperature. The mixture is evaporated to dryness using the rotary evaporator. The residue is diluted with water and extracted three times with ethyl acetate. The combined organic phases are dried on sodium sulfate and evaporated to dryness in vacuo. A white solid is obtained. Yield: 360 mg (90%); R. value: 2.25 min; C₂₂H₂₅N₃O₃ (379.45); mass spectrum: (M+H)⁺=380.

### (d) benzyl (R)-[1-(3-methyl-2,3,4,5-tetrahydro-1H-benzo[d]azepin-7-yl)-5-oxopyrrolidin-3-yl]carbamate

320 mg (0.84 mmol) of benzyl (R)-[5-oxo-1-(2,3,4,5-tetrahydro-1H-benzo[d]azepin-7-yl)pyrrolidin-3-yl]carbamate is suspended in 2 mL of methanol and acidified with glacial acetic acid (pH=6). 120 μL (1.6 mmol) of an aqueous formaldehyde solution (37%) is added and the mixture is stirred for 30 minutes at ambient temperature. Then 340 mg (1.6 mmol) of sodium triacetoxyborohydride is added. After an hour, the mixture is poured onto saturated sodium hydrogen carbonate solution and extracted three times with ethyl acetate. The combined organic phases are dried on sodium sulfate and evaporated to dryness in vacuo. A white solid is obtained. Yield: 320 mg (96%); R. value: 2.25 min; C₂₃H₂₇N₃O₃ (393.48); mass spectrum: (M+H)⁺=394.

### (e) (R)-4-amino-1-(3-methyl-2,3,4,5-tetrahydro-1H-benzo[d]azepin-7-yl]pyrrolidin-2-one

320 mg (0.8 mmol) of benzyl (R)-[1-(3-methyl-2,3,4,5-tetrahydro-1H-benzo[d]azepin-7-yl)-5-oxopyrrolidin-3-yl]carbamate is dissolved in a mixture of 10 mL of methanol and 6 mL of THF, combined with 100 mg palladium on charcoal and hydrogenated in a Parr apparatus at 1 bar hydrogen pressure at ambient temperature for 45 minutes. The mixture is filtered off from the catalyst and evaporated to dryness using the rotary evaporator. Yield: 210 mg (quantitative); R_{f} value: 0.05 (silica gel; dichloromethane/methanol 90:10); C₁₅H₂₁N₃O (259.35); mass spectrum: (M+H)⁺=260.

### (f) (R)-5-bromothiophene-2-carboxylic acid-[1-(3-methyl-2,3,4,5-tetrahydro-1H-benzo[d]azepin-7-yl)-5-oxopyrrolidin-3-yl]amide (monotrifluoroacetate salt)

72 mg (0.35 mmol) of 5-bromothiophene-2-carboxylic acid is combined in 1.5 mL of DMF with 120 μL (1.1 mmol) of NMM and 113 mg (0.35 mmol) of TBTU and then stirred for 30 minutes under a nitrogen atmosphere at ambient temperature. Then 92 mg (0.35 mmol) of (R)-4-amino-1-(3-methyl-2,3,4,5-tetrahydro-1H-benzo[d]azepin-7-yl]pyrrolidin-2-one dissolved in 0.5 mL of DMF are added and the mixture is stirred for 16 hours at ambient temperature. The reaction mixture is then acidified with trifluoroacetic acid and purified by reversed-phase chromatography. Yield: 86 mg (44%); Rₜ value: 2.34 min; C₂₀H₂₂BrN₃O₂S (448.39); mass spectrum: (M+H)⁺=448/450 (bromine isotopes).

The following compounds were prepared analogously:

| No. | Structural formula Name | Yield | Mass peak(s) | R_{f} value or Rₜ |
|---|---|---|---|---|
| 2 | 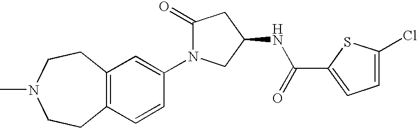 | Σ: 5.4% | (M − H)⁻ = 404/406 (chlorine isotopes) | 2.33 min |
| | (R)-5-chlorothiophene-2-carboxylic acid-[1-(3-methyl-2,3,4,5-tetrahydro-1H- | | | |
| | benzo[d]azepin-7-yl)-5-oxopyrrolidin-3-yl]amide (monotrifluoroacetate salt) | | | |
| 4 | 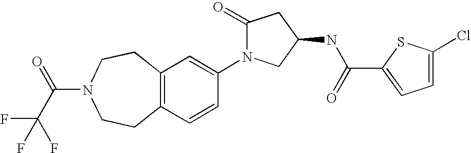 | Σ: 14.9% | (M + H)⁺ = 486/488 (chlorine isotopes) | 3.17 min |
| | (R)-5-chlorothiophene-2-carboxylic acid-{5-oxo-1-[3-(2,2,2-trifluoroacetyl)-2,3,4,5- | | | |
| | tetrahydro-1H-benzo[d]azepin-7-yl]-pyrrolidin-3-yl}amide | | | |
| 5 | 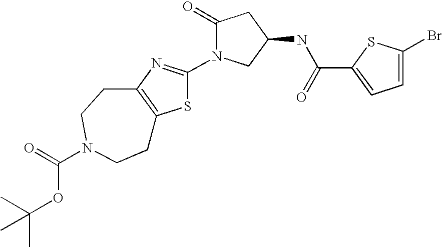 | Σ: 4.3% | (M + H)⁺ = 541/543 (bromine isotopes) | 3.32 min |
| | tert-butyl (R)-2-{4-[(5-bromothiophene-2-carbonyl)amino]-2-oxopyrrolidine-1-yl}- | | | |
| | 4,5,7,8-tetrahydrothiazolo[4,5-d]azepine-6-carboxylate | | | |
| 19 | 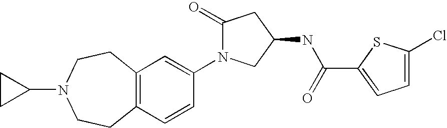 | Σ: 4.3% | (M + H)⁺ = 430/432 (chlorine isotopes) | 4.03 min |

(R)-5-chlorothiophenecarboxylic acid-[1-(3-cyclopropyl-2,3,4,5-tetrahydro-1H-
benzo[d]azepin-7-yl)-5-oxopyrrolidin-3-yl]amide (monotrifluoroacetate salt)

### EXAMPLE 3

### 5-chlorothiophene-2-carboxylic acid-[(3R,4R)-1-(3-methyl-2,3,4,5-tetrahydro-1H-[d]azepin-7-yl)-5-oxo-4-propylpyrrolidin-3-yl]amide (monotrifluoroacetate salt)

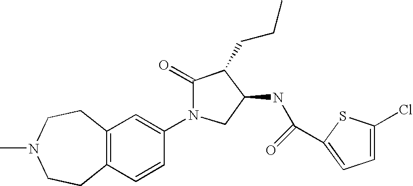

### (a) N-(2,3,4,5-tetrahydro-1H-benzo[d]azepin-7-yl)acetamide

15.6 g (61.4 mmol) of 7-amino-3-trifluoroacetyl-2,3,4,5-tetrahydro-1H-benzo[d]azepine is dissolved in 50 mL of glacial acetic acid, combined with 8.7 mL (92 mmol) of acetic anhydride and stirred for 16 hours at ambient temperature. Then the mixture is poured onto water and the precipitated solid is filtered off and rinsed with copious amounts of water. The still fairly moist solid is dissolved in a mixture of 400 mL of methanol, 100 mL of water, and 50 mL of THF, combined with 30 g (215 mmol) of potassium carbonate and stirred for one hour at ambient temperature. Then the organic solvents are eliminated using the rotary evaporator; the aqueous residue is diluted with 100 mL of water and extracted three times with dichloromethane. The combined organic phases are dried on sodium sulfate and concentrated in vacuo. A yellowish-orange oil is obtained. Yield: 9 g (74%); R_{f} value: 0.12 (silica gel; dichloromethane/methanol 90:10); C₁₂H₁₆N₂O (204.27); mass spectrum: (M+H)⁺=205.

### (b) N-(3-methyl-2,3,4,5-tetrahydro-1H-benzo[d]azepin-7-yl)acetamide

Prepared analogously to Example 1d from N-(2,3,4,5-tetrahydro-1H-benzo[d]azepin-7-yl)-acetamide by reductive alkylation with formaldehyde solution. Yield: 91%; R_{f} value: 0.35 (silica gel; dichloromethane/methanol 70:30); C₁₃H₁₈N₂O (218.30); mass spectrum: (M+H)⁺=219.

### (c) 3-methyl-2,3,4,5-tetrahydro-1H-benzo[d]azepin-7-ylamine (as the bis-hydrochloride salt)

2.75 g (12.6 mmol) of N-(3-methyl-2,3,4,5-tetrahydro-1H-benzo[d]azepin-7-yl)acetamide is dissolved in 20 mL of semi-concentrated hydrochloric acid and irradiated for 45 minutes in the microwave oven at 260 Watt. Then the mixture is evaporated to dryness. A slightly reddish, glassy solid is obtained. Yield: 91%; R_{f} value: 0.09 (silica gel; dichloromethane/methanol 70:30); C₁₃H₁₈N₂O (176.26); mass spectrum: (M+H)⁺=177.

### (d) benzyl[(3R,4R)-4-allyl-5-oxotetrahydrofuran-3-yl]carbamate

2.72 mL (19.4 mmol) of diisopropylamine is placed in 30 mL of THF and combined with 12 mL (19.2 mmol) of n-butyl lithium solution in hexane (1.6M) while cooling with ice. The mixture is stirred for 10 minutes at 0° C., then cooled to −78° C. and a solution of 2 g (8.5 mmol) of benzyl (R)-(5-oxotetrahydrofuran-3-yl)carbamate in 10 mL of THF is added dropwise. The mixture is stirred for one hour at −78° C. Then 2.86 mL (33 mmol) of allyl bromide is added dropwise, and the mixture is heated to −60° C. within one hour. Then 5 mL of saturated ammonium chloride solution is added and the mixture is heated to ambient temperature. Water is added and the mixture is extracted three times with ethyl acetate. The combined organic phases are dried on sodium sulfate and evaporated to dryness. The residue is taken up in DMF, acidified with TFA, and purified by reversed-phase chromatography. Yield: 820 mg (35%); Rₜ value: 2.70 min; C₁₅H₁₇NO₄ (275.30); mass spectrum: (M−H)⁻=276.

### (e) benzyl {(1R,2R)-1-hydroxymethyl-2-(3-methyl-2,3,4,5-tetrahydro-1H-benzo[d]azepin-7-ylcarbamoyl)pent-4-enyl}carbamate

Prepared analogously to Example 1a from 3-methyl-2,3,4,5-tetrahydro-1H-benzo[d]azepin-7-ylamine-dihydrochloride and benzyl [(3R,4R)-4-allyl-5-oxotetrahydrofuran-3-yl]carbamate with trimethylaluminum in dichloromethane. Purification is carried out by reversed-phase chromatography. Yield: 50%; Rt value: 4.04 min; C₂₆H₃₃N₃O₄ (451.56); mass spectrum: (M+H)⁺=451.

### (f) benzyl {(3R,4R)-4-allyl-1-(3-methyl-2,3,4,5-tetrahydro-1H-benzo[d]azepin-7-yl)-5-oxopyrrolidin-3-yl}carbamate (as the monohydrochloride salt)

Prepared analogously to Example 1b from benzyl {(1R,2R)-1-hydroxymethyl-2-(3-methyl-2,3,4,5-tetrahydro-1H-benzo[d]azepin-7-ylcarbamoyl)pent-4-enyl}carbamate with di-tert-butylazodicarboxylate and tributylphosphine in THF. Purification is carried out by reversed-phase chromatography. The residue is dissolved in 1N hydrochloric acid and extracted three times with ethyl acetate. The aqueous phase is freeze-dried. A colorless solid is obtained. Yield: 21% (purity 50%); R. value: 4.04 min; C₂₆H₃₁N₃O₃ (433.56); mass spectrum: (M+H)⁺=434.

### (g) (3R,4R)-4-amino-1-(3-methyl-2,3,4,5-tetrahydro-1H-benzo[d]azepin-7-yl)-3-propylpyrrolidin-2-one (as the bis-hydrochloride salt)

Prepared analogously to Example 1e from benzyl {(3R,4R)-4-allyl-1-(3-methyl-2,3,4,5-tetrahydro-1H-benzo[d]azepin-7-yl)-5-oxopyrrolidin-3-yl}carbamate hydrochloride. Yield: 35% (purity 50%); Rt value: 3.02 min; C₁₈H₂₇N₃O (301.44); mass spectrum: (M+H)⁺=302.

### (h) 5-chlorothiophene-2-carboxylic acid-[(3R,4R)-1-(3-methyl-2,3,4,5-tetrahydro-1H-benzo[d]azepin-7-yl)-5-oxo-4-propylpyrrolidin-3-yl]amide (as the monotrifluoroacetate salt)

Prepared analogously to Example 1f from (3R,4R)-4-amino-1-(3-methyl-2,3,4,5-tetrahydro-1H-benzo[d]azepin-7-yl)-3-propylpyrrolidin-2-one-dihydrochloride and 5-chlorothiophene-2-carboxylic acid with TBTU and NMM. The reaction mixture is evaporated down, taken up in diethyl ether/isopropanol, and combined with ethereal hydrochloric acid solution. The precipitate is washed twice with diethyl ether, then taken up in a water/trifluoroacetic acid/acetonitrile mixture, and purified by reversed-phase chromatography. Yield: 79%; Rₜ value: 4.59 min; C₂₃H₂₈ClN₃O₂S (446.02); mass spectrum: (M+H)⁺=446/448 (chlorine isotopes).

The following compounds were prepared analogously:

| No. | Structural formula Name | Yield | Mass peak(s) | R_{f} value or Rₜ |
|---|---|---|---|---|
| 11 | 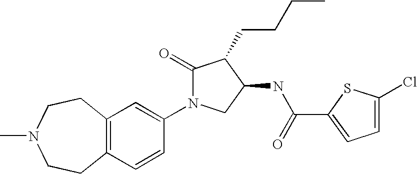 | Σ: 5.4% | (M + H)⁺ = 460/462 (chlorine isotopes) | 2.74 min |
| | 5-chlorothiophene-2-carboxylic acid-[(3R,4R)-4-butyl-1-(3-methyl-2,3,4,5-tetrahydro- | | | |
| | 1H-benzo[d]azepin-7-yl)-5-oxopyrrolidin-3-yl]amide (monotrifluoroacetate salt) | | | |
| 12 | 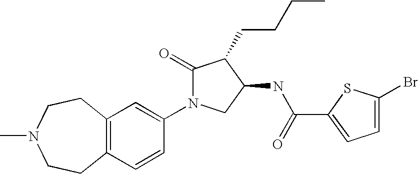 | Σ: 5.4% | (M + H)⁺ = 504/506 (bromine isotopes) | 2.76 min |
| | 5-bromothiophene-2-carboxylic acid-[(3R,4R)-4-butyl-1-(3-methyl-2,3,4,5- | | | |
| | tetrahydro-1H-benzo[d]azepin-7-yl)-5-oxopyrrolidin-3-yl]amide | | | |
| | (monotrifluoroacetate salt) | | | |
| 14 | 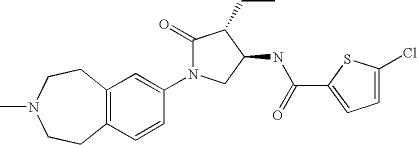 | Σ: 15.9% | (M + H)⁺ = 432/434 (chlorine isotopes) | 2.51 min |
| | 5-chlorothiophene-2-carboxylic acid-[(3R,4R)-4-ethyl-1-(3-methyl-2,3,4,5-tetrahydro- | | | |
| | 1H-benzo[d]azepin-7-yl)-5-oxopyrrolidin-3-yl]amide (monotrifluoroacetate salt) | | | |
| 20 | 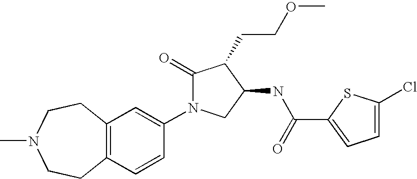 | Σ: 15.9% | (M + H)⁺ = 462/464 (chlorine isotopes) | 2.49 min |
| | 5-chlorothiophene-2-carboxylic acid-[(3R,4R)-4-(2-methoxyethyl)-1-(3-methyl- | | | |
| | 2,3,4,5-tetrahydro-1H-benzo[d]azepin-7-yl)-5-oxopyrrolidin-3-yl]amide | | | |
| | (monotrifluoroacetate salt) | | | |
| 21 | 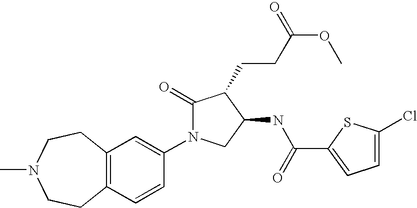 | Σ: 0.2% | (M + H)⁺ = 504/506 (chlorine isotopes) | 2.61 min |
| | 5-chlorothiophene-2-carboxylic acid-[(3R,4R)-4-(3-methoxycarbonylpropyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 21 | 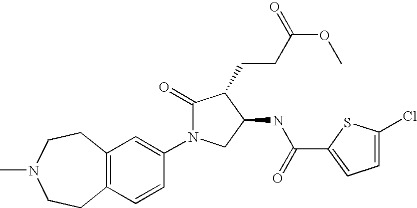 | Σ: 0.2% | (M + H)⁺ = 504/506 (chlorine isotopes) | 2.61 min |
| | 5-chlorothiophene-2-carboxylic acid-[(3R,4R)-4-(3-methoxycarbonylpropyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 22 | 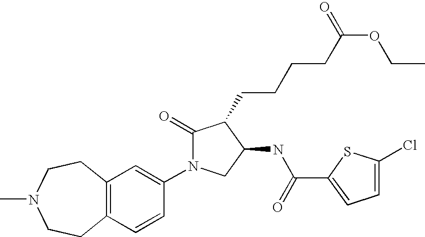 | Σ: 0.2% | (M + H)⁺ = 532/534 (chlorine isotopes) | 2.77 min |
| | 5-chlorothiophene-2-carboxylic acid-[(3R,4R)-4-(3-ethoxycarbonyl-butyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 22 | 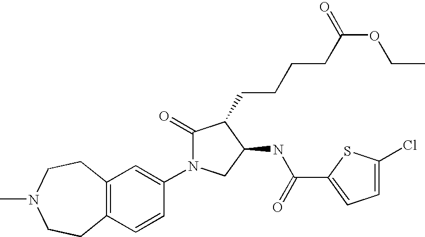 | Σ: 0.2% | (M + H)⁺ = 532/534 (chlorine isotopes) | 2.77 min |
| | 5-chlorothiophene-2-carboxylic acid-[(3R,4R)-4-(3-ethoxycarbonyl-butyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 23 | 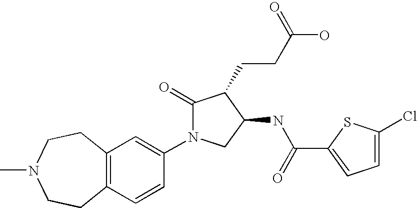 | Σ: 0.2% | (M + H)⁺ = 490/492 (chlorine isotopes) | 3.94 min |
| | 5-chlorothiophene-2-carboxylic acid-[(3R,4R)-4-(3-hydroxycarbonylpropyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 23 | 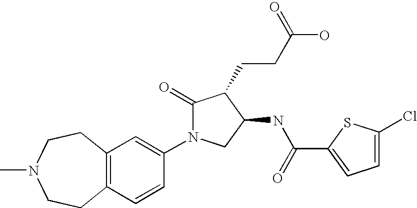 | Σ: 0.2% | (M + H)⁺ = 490/492 (chlorine isotopes) | 3.94 min |
| | 5-chlorothiophene-2-carboxylic acid-[(3R,4R)-4-(3-hydroxycarbonylpropyl)-1-(3- | | | |
| | methyl-2,3,4,5-tetrahydro-1H-benzo[d]azepin-7-yl)-5-oxopyrrolidin-3-yl]amide | | | |
| | (monotrifluoroacetate salt) | | | |
| 24 | 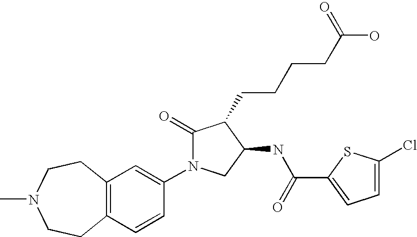 | Σ: 0.2% | (M + H)⁺ = 504/506 (chlorine isotopes) | 2.46 min |

5-chlorothiophene-2-carboxylic acid-[(3R,4R)-4-(3-hydroxycarbonyl-butyl)-1-(3-
methyl-2,3,4,5-tetrahydro-1H-benzo[d]azepin-7-yl)-5-oxopyrrolidin-3-yl]amide
(monotrifluoroacetate salt)

### EXAMPLE 6

### (R)-5-chlorothiophene-2-carboxylic acid-[5-oxo-1-(2,3,4,5-tetrahydro-1H-benzo[d]azepin-7-yl)pyrrolidin-7-ylamine

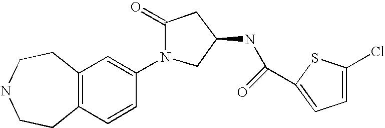

### (a) (R)-5-chlorothiophene-2-carboxylic acid-[5-oxo-1-(2,3,4,5-tetrahydro-1H-benzo[d]azepin-7-yl)pyrrolidin-7-yl]amide

Prepared analogously to Example 1c from (R)-5-chlorothiophene-2-carboxylic acid-{5-oxo-1-[3-(2,2,2-trifluoroacetyl)-2,3,4,5-tetrahydro-1H-benzo[d]azepin-7-yl]pyrrolidin-3-yl}amide. A colorless solid is obtained. Yield: 221 mg (78%); Rₜ value: 2.37 min; C₁₉H₂₀ClN₃O₂S (389.90); mass spectrum: (M+H)⁺=390/392 (chlorine isotopes).

### EXAMPLE 7

### (R)-5-bromothiophene-2-carboxylic acid-[5-oxo-1-(5,6,7,8-tetrahydro-4H-thiazolo[4,5-d]azepin-2-yl)pyrrolidin-3-yl]amide (monotrifluoroacetate salt)

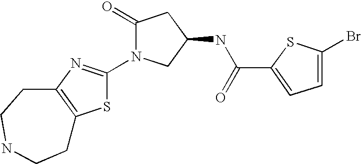

### (a) (R)-5-bromothiophene-2-carboxylic acid-[5-oxo-1-(5,6,7,8-tetrahydro-4H-thiazolo[4,5-d]azepin-2-yl)pyrrolidin-3-yl]amide (monotrifluoroacetate salt)

50 mg (92 μmol) of tert-butyl (R)-2-{4-[(5-bromothiophene-2-carbonyl)amino]-2-oxopyrrolidin-1-yl}-4,5,7,8-tetrahydrothiazolo[4,5-d]azepine-6-carboxylate is dissolved in a mixture of 0.5 mL of dichloromethane and 0.25 mL of trifluoroacetic acid and stirred for one hour at ambient temperature. Then the mixture is evaporated to dryness, dissolved in water, and lyophilized. A colorless solid is obtained. Yield: 44 mg (85%); Rₜ value: 2.37 min; C₁₆H₁₇BrN₄O₂S₂ (441.37); mass spectrum: (M+H)⁺=441/443 (bromine isotopes).

### EXAMPLE 8

### (R)-5-chlorothiophene-2-carboxylic acid-[1-(3-ethyl-2,3,4,5-tetrahydro-1H-benzo[d]azepin-7-yl)-5-oxopyrrolidin-3-yl]amide

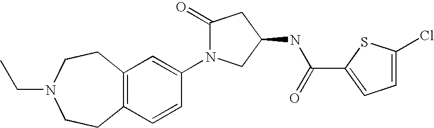

### (a) (R)-5-chlorothiophene-2-carboxylic acid-[1-(3-ethyl-2,3,4,5-tetrahydro-1H-benzo[d]azepin-7-yl)-5-oxopyrrolidin-3-yl]amide

100 g (256 μmol) of (R)-5-chlorothiophene-2-carboxylic acid-[5-oxo-1-(2,3,4,5-tetrahydro-1H-benzo[d]azepin-7-yl)pyrrolidin-7-yl]amide is dissolved in a mixture of 1.5 mL of THF and 1 mL of DMF and cooled to 0° C. 36 μL (256 μmol) of triethylamine and then 23 μL (282 μmol) of ethyl iodide are added dropwise and the mixture is stirred at ambient temperature for 18 hours. Then the mixture is evaporated to dryness. A yellowish solid is obtained. Yield: 101 mg (95%); Rₜ value: 2.36 min; C₂₁H₂₄ClN₃O₂S (417.95); mass spectrum: (M−H)⁻=416/418 (chlorine isotopes).

The following compounds were prepared analogously:

| No. | Structural Formula Name | Yield | Mass peak(s) | R_{f} value or Rₜ |
|---|---|---|---|---|
| 9 | 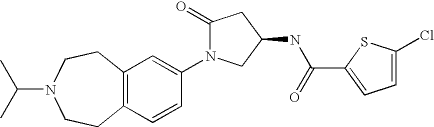 | Σ: 54% | (M + H)⁺ = 432/434 (chlorine isotopes) 2.40 min | |
| | (R)-5-chlorothiophene-2-carboxylic acid-[1-(3-isopropyl-2,3,4,5-tetrahydro-1H- | | | |
| | benzo[d]azepin-7-yl)-5-oxopyrrolidin-3-yl]amide (monotrifluoroacetate salt) | | | |
| 10 | 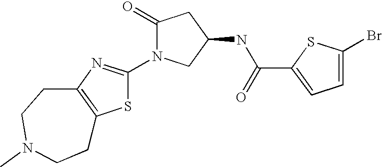 | Σ: 29% | (M + H)⁺ = 455/457 (bromine isotopes) 2.33 min | |
| | (R)-5-bromothiophene-2-carboxylic acid-[1-(6-methyl-5,6,7,8-tetrahydro-4H- | | | |
| | thiazolo[4,5-d]azepin-2-yl)-5-oxopyrrolidin-3-yl]amide (monotrifluoroacetate salt) | | | |
| 16 | 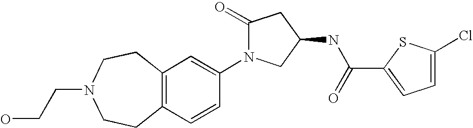 | Σ: 94% | (M + H)⁺ = 434/436 (chlorine isotopes) 2.34 min | |
| | (R)-5-chlorothiophene-2-carboxylic acid-{1-[3-(2-hydroxyethyl)-2,3,4,5-tetrahydro- | | | |
| | 1H-benzo[d]azepin-7-yl]-5-oxopyrrolidin-3-yl}amide (monotrifluoroacetate salt) | | | |
| 17 | 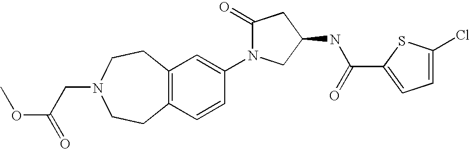 | Σ: 97% | (M + H)⁺ = 462/464 (chlorine isotopes) 2.42 min | |
| | (R)-5-chlorothiophene-2-carboxylic acid-[1-(3-methoxycarbonylmethyl-2,3,4,5- | | | |
| | tetrahydro-1H-benzo[d]azepin-7-yl)-5-oxopyrrolidin-3-yl]amide | | | |
| | (monotrifluoroacetate salt) | | | |
| 18 | 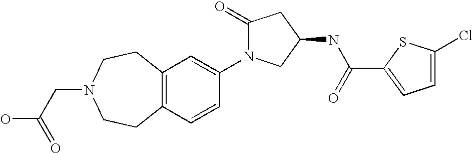 | Σ: 45% | (M + H)⁺ = 448/450 (chlorine isotopes) 2.29 min | |

(R)-5-chlorothiophene-2-carboxylic acid-[1-(3-hydroxycarbonylmethyl-2,3,4,5-
tetrahydro-1H-benzo[d]azepin-7-yl)-5-oxopyrrolidin-3-yl]amide
(monotrifluoroacetate salt)

### EXAMPLE 13

### 5-bromothiophene-2-carboxylic acid-[1-(3-methyl-2,3,4,5-tetrahydro-1H-benzo[d]azepin-7-yl)-5-oxopyrrolidin-3-ylmethyl]amide (monotrifluoroacetate salt)

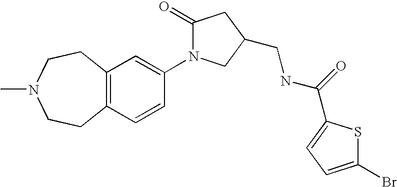

### (a) 1-(3-methyl-2,3,4,5-tetrahydro-1H-benzo[d]azepin-7-yl)-5-oxopyrrolidine-3-carboxylic acid (monotrifluoroacetate salt)

895 mg (3.59 mmol) of 3-methyl-2,3,4,5-tetrahydrobenzo[d]azepin-7-ylamine-bis-hydrochloride is suspended in 3 mL of DCM and combined with 0.61 mL (3.59 mmol) of diisopropylethylamine. The mixture is stirred for five minutes and then 701 mg (5.39 mmol) of itaconic acid is added. This mixture is heated to 50° C. with stirring until the solvent has evaporated, and then melted for 4.5 hours at 140° C. Then the melt is cooled, dissolved in a mixture of water and trifluoroacetic acid and chromatographically purified by RP-HPLC. A colorless solid is obtained. Yield: 764 mg (53%); Rₜ value: 1.71 min; C₁₆H₂₀N₂O₃ (288.35); mass spectrum: (M+H)⁺=289.

### (b) 4-hydroxymethyl-1-(3-methyl-2,3,4,5-tetrahydrobenzo[d]azepin-7-yl)pyrrolidin-2-one (monotrifluoroacetate salt)

500 mg (1.24 mmol) of 1-(3-methyl-2,3,4,5-tetrahydro-1H-benzo[d]azepin-7-yl)-5-oxopyrrolidine-3-carboxylic acid-monotrifluoroacetate is suspended in 20 mL of THF and at 0° C. combined with 9 mL (9 mmol) of borane-THF complex (1M in THF). The mixture is stirred for 20 hours at RT and the solvent is distilled off. The residue is suspended in water and trifluoroacetic acid and stirred until homogeneous. Then the aqueous phase is extracted three times with ethyl acetate. The combined organic phases are dried on sodium sulfate and evaporated down. The residue is chromatographically purified by RP-HPLC. A colorless oil is obtained. Yield: 80 mg (17%); Rₜ value: 1.60 min; C₁₆H₂₂N₂O₂ (274.37); mass spectrum: (M+H)⁺=275.

### (c) 1-(3-methyl-2,3,4,5-tetrahydrobenzo[d]azepin-7-yl)-5-oxopyrrolidin-3-ylmethyl methanesulfonate

79 mg (203 μmol) of 4-hydroxymethyl-1-(3-methyl-2,3,4,5-tetrahydrobenzo[d]azepin-7-yl)pyrrolidin-2-one-monotrifluoroacetate is dissolved in 10 mL of DCM and combined with 150 μL (1.1 mmol) of triethylamine. The mixture is cooled to 0° C., combined with 50 μL (646 μmol) of methanesulfonyl chloride and stirred at RT for five hours. The solution is combined with water and the aqueous phase is extracted three times with ethyl acetate. The combined organic phases are dried on sodium sulfate and evaporated down. The methanesulfonate is obtained as the crude product (66 mg, orange oil). Rₜ value: 1.90 min; C₁₇H₂₄N₂O₄S (352.76); mass spectrum: (M+H)⁺=353.

### (d) 4-azidomethyl-1-(3-methyl-2,3,4,5-tetrahydro-1H-benzo[d]azepin-7-yl)pyrrolidin-2-one

66 mg of 1-(3-methyl-2,3,4,5-tetrahydrobenzo[d]azepin-7-yl)-5-oxopyrrolidin-3-ylmethyl methanesulfonate is dissolved as the crude product in 5 mL of DMF and combined with 40 mg (615 μmol) of sodium azide. The mixture is stirred for 20 hours at 50° C. and the solvent is distilled off under a slight underpressure using the rotary evaporator. The residue is taken up in ethyl acetate and washed with a mixture of saturated sodium hydrogen carbonate solution and saturated saline solution. The aqueous phase is extracted three times more with ethyl acetate. The combined organic phases are dried on sodium sulfate and evaporated down. The azide is obtained as a crude product (56 mg, orange oil). Rₜ value: 2.08 min; C₁₆H₂₁N₅O (299.37); mass spectrum: (M+H)⁺=300.

### (e) 5-bromothiophene-2-carboxylic acid-[1-(3-methyl-2,3,4,5-tetrahydro-1H-benzo[d]azepin-7-yl)-5-oxopyrrolidin-3-ylmethyl]amide (monotrifluoroacetate salt)

56 mg of 4-azidomethyl-1-(3-methyl-2,3,4,5-tetrahydro-1H-benzo[d]azepin-7-yl)pyrrolidin-2-one is hydrogenated as the crude product in methanol analogously to Example 1e. The amine thus obtained (crude product, 25 mg, colorless oil) is dissolved in DMF and reacted analogously to Example 1f with 5-bromothiophene-2-carboxylic acid, TBTU, and NMM. Purification is carried out chromatographically by RP-HPLC. A colorless solid is obtained. Yield: 8.4% (starting from 4-hydroxymethyl-1-(3-methyl-2,3,4,5-tetrahydrobenzo[d]azepin-7-yl)pyrrolidin-2-one (monotrifluoroacetate salt)); Rₜ value: 2.35 min; C₂₁H₂₄BrN₃O₂S (462.41); mass spectrum: (M+H)⁺=462/464 (bromine isotopes).

### EXAMPLE 15

### (R)-5-chlorothiophene-2-thiocarboxylic acid-[1-(3-ethyl-2,3,4,5-tetrahydro-1H-benzo[d]azepin-7-yl)-5-thioxopyrrolidin-3-ylmethyl]amide (monotrifluoroacetate salt)

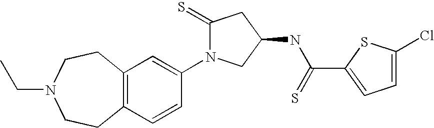

### (a) 1-(3-methyl-2,3,4,5-tetrahydro-1H-benzo[d]azepin-7-yl)-5-oxopyrrolidine-3-carboxylic acid (monotrifluoroacetate salt)

60 mg (144 μmol) of (R)-5-chlorothiophene-2-carboxylic acid-[1-(3-ethyl-2,3,4,5-tetrahydro-1H-benzo[d]azepin-7-yl)-5-oxopyrrolidin-3-yl]amide is dissolved in 2 mL of dioxane and combined with 116 mg (288 μmol) of Lawesson's reagent. The mixture is stirred for two hours at reflux temperature. Then the mixture is cooled and the volatile constituents are eliminated in vacuo. The residue is dissolved in a mixture of water and trifluoroacetic acid and purified chromatographically by RP-HPLC. Yield: 67 mg (81%); Rₜ value: 2.85 min; C₂₁H₂₄ClN₃S₃ (450.09); mass spectrum: (M+H)⁺=450/452 (chlorine isotopes).

Each of the patents, patent applications, or other references cited herein is hereby incorporated by reference in its entirety.

### EXAMPLE A

### Dry Ampoule Containing 75 mp of Active Substance per 10 mL

Composition:

| | |
|---|---|
| Active substance | 75.0 mg |
| Mannitol | 50.0 mg |
| water for injections | ad 10.0 mL |

Preparation: Active substance and mannitol are dissolved in water. After packaging, the solution is freeze-dried. To produce the solution ready for use for injections, the product is dissolved in water.

### EXAMPLE B

### Dry Ampoule Containing 35 mg of Active Substance per 2 mL

Composition:

| | |
|---|---|
| Active substance | 35.0 mg |
| Mannitol | 100.0 mg |
| water for injections | ad 2.0 mL |

Preparation: Active substance and mannitol are dissolved in water. After packaging, the solution is freeze-dried. To produce the solution ready for use for injections, the product is dissolved in water.

### EXAMPLE C

### Tablet Containing 50 mg of Active Substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation: (1), (2), and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### EXAMPLE D

### Tablet Containing 350 mg of Active Substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation: (1), (2), and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### EXAMPLE E

### Capsules Containing 50 mg of Active Substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation: (1) is triturated with (3). This trituration is added to the mixture of (2) and (4) with vigorous mixing. This powder mixture is packed into size 3 hard gelatine capsules in a capsule filling machine.

### EXAMPLE F

### Capsules Containing 350 mg of Active Substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation: (1) is triturated with (3). This trituration is added to the mixture of (2) and (4) with vigorous mixing. This powder mixture is packed into size 0 hard gelatine capsules in a capsule filling machine.

### EXAMPLE G

### Suppositories Containing 100 mg of Active Substance

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Preparation: The polyethyleneglycol is melted together with polyethylenesorbitan monostearate. At 40° C. the ground active substance is homogeneously dispersed in the melt. It is cooled to 38° C. and poured into slightly chilled suppository moulds.

We claim:

1. A compound of formula (I) 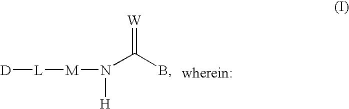
D is a substituted bicyclic ring system of formula (II) 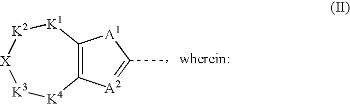
K¹ and K⁴ are each independently a —CH₂—, —CHR^{7a}—, —CR^{7b}R^{7c}—, or a —C(O)— group, wherein R^{7a}/R^{7b}/R^{7c} are each independently a fluorine atom, a hydroxy, C₁₋₅-alkoxy, amino, C₁₋₅-alkylamino, di-(C₁₋₅-alkyl)-amino, C₃₋₅-cycloalkyleneimino, C₁₋₅-alkylcarbonylamino group, a C₁₋₅-alkyl group optionally substituted by one to three fluorine atoms, a hydroxy-C₁₋₅-alkyl, C₁₋₅-alkoxy-C₁₋₅-alkyl, amino-C₁₋₅-alkyl, C₁₋₅-alkylamino-C₁₋₅-alkyl, di-(C₁₋₅-alkyl)-amino-C₁₋₅-alkyl, C₄₋₇-cycloalkyleneimino-C₁₋₅-alkyl, carboxy-C₀₋₅-alkyl, C₁₋₅-alkoxycarbonyl-C₀₋₅-alkyl, aminocarbonyl-C₀₋₅-alkyl, C₁₋₅-alkylaminocarbonyl-C₀₋₅-alkyl, di-(C₁₋₅-alkyl)-aminocarbonyl-C₀₋₅-alkyl, or C₄₋₇-cycloalkyleneiminocarbonyl-C₀₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom form a 3-, 4-, 5-, 6-, or 7-membered saturated carbocycle, or a cyclopentene, cyclohexene, oxetan, azetidine, thietan, tetrahydrofuran, pyrrolidine, tetrahydrothiophene, tetrahydropyran, piperidine, pentamethylene sulfide, hexamethyleneimine, 1,3-dioxolane, 1,4-dioxane, hexahydropyridazine, piperazine, thiomorpholine, morpholine, 2-imidazolidinone, 2-oxazolidinone, tetrahydro-2(1H)-pyrimidinone, or [1,3]oxazinan-2-one ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides an N atom is optionally replaced by a —C(O)— group, and/or the imino groups of which are each optionally substituted by a C₁₋₃-alkyl or C₁₋₃-alkylcarbonyl group, and/or wherein the sulfur atom is optionally oxidized to form a sulfoxide or sulfone group;
K² and K³ are each independently a —CH₂—, —CHR^{8a}—, —CR^{8b}R^{8c}, or a —C(O)— group, wherein R^{8a}/R^{8b}/R^{8c} are each independently a C₁₋₅-alkyl group optionally substituted by one to three fluorine atoms, a hydroxy-C₁₋₅-alkyl, C₁₋₅-alkoxy-C₁₋₅-alkyl, amino-C₁₋₅-alkyl, C₁₋₅-alkyl-amino-C₁₋₅-alkyl, di-(C₁₋₅-alkyl)-amino-C₁₋₅-alkyl, C₄₋₇-cyclo-alkyleneimino-C₁₋₅-alkyl, carboxy-C₁₋₅-alkyl, C₁₋₅-alkoxycarbonyl-C₁₋₅-alkyl, aminocarbonyl-C₁₋₅-alkyl, C₁₋₅-alkylaminocarbonyl-C₁₋₅-alkyl, di-(C₁₋₅-alkyl)-aminocarbonyl-C₁₋₅-alkyl, or C₄₋₇-cycloalkyleneiminocarbonyl-C₁₋₅-alkyl group, or two groups R^{8b}/R^{8c} together with the cyclic carbon atom form a 3-, 4-, 5-, 6-, or 7-membered saturated carbocycle or a cyclopentene, cyclohexene, oxetan, azetidine, thietan, tetrahydrofuran, pyrrolidine, tetrahydrothiophene, tetrahydropyran, piperidine, pentamethylene sulfide, hexamethyleneimine, hexahydropyridazine, tetrahydro-2(1H)-pyrimidinone, or [1,3]oxazinan-2-one ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides a nitrogen atom is optionally replaced by a —CO— group, and/or the imino groups of which are each optionally substituted by a C₁₋₃-alkyl or C₁₋₃-alkylcarbonyl group, and/or wherein the sulfur atom is optionally oxidized to form a sulfoxide or sulfone group, with the proviso that a heteroatom introduced by R^{8b}or R^{8c} cannot be separated from X in formula I by only one carbon atom,
wherein in total formula (II) contains a maximum of four groups selected from R^{7a}, R^{7b}, R^{7c}, R^{8a}, R^{8b}, and R^{8c};
X is an oxygen or sulfur atom, a sulfene, sulfone, or —N(R¹)— group, wherein R¹ is a hydrogen atom or a hydroxy, C₁₋₃-alkoxy, amino, C₁₋₃-alkylamino, di-(C₁₋₃-alkyl)-amino, C₁₋₅-alkyl, C₃₋₅-alkenyl-CH₂, C₃₋₅-alkynyl-CH₂, C₃₋₆-cycloalkyl, C₄₋₆-cycloalkenyl, oxetan-3-yl, tetrahydrofuran-3-yl, benzyl, C₁₋₅-alkylcarbonyl, trifluoromethylcarbonyl, C₃₋₆-cycloalkylcarbonyl, C₁₋₅-alkylsulfonyl, C₃₋₆-cycloalkylsulfonyl, aminocarbonyl, C₁₋₅-alkylaminocarbonyl, di-(C₁₋₅-alkyl)-aminocarbonyl, C₁₋₅-alkyloxycarbonyl, or C₄₋₇-cycloalkyleneiminocarbonyl group, wherein the methylene and methyl groups present in the groups mentioned previously are optionally additionally substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group, provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is an oxygen or sulfur atom, a —C(R¹⁰)═N—, —N═C(R¹⁰), or —C(R¹⁰)═C(R¹¹)— group,
A² is either a nitrogen atom or a ═C(R¹²)— group, wherein R¹⁰, R¹¹, and R¹² are each independently a hydrogen, fluorine, chlorine, bromine, or iodine atom, or a C₁₋₅-alkyl, —CF₃, C₂₋₅-alkenyl, C₂₋₅-alkynyl, cyano, carboxy, C₁₋₅-alkoxycarbonyl, hydroxy, C₁₋₃-alkoxy, CF₃O—, CHF₂O—, CH₂FO—, amino, C₁₋₅-alkylamino, di-(C₁₋₅-alkyl)-amino, or C₄₋₇-cycloalkyleneimino group;
L is a substituted ring system of formula (IIa) or (IIb) 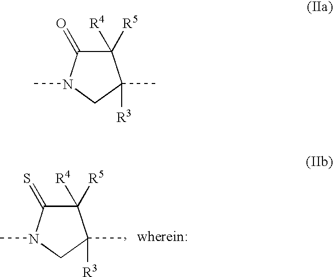
R³ is a hydrogen atom or a C₁₋₃-alkyl group;
R⁴ and R⁵ are each independently a hydrogen atom, a hydroxy group, an —OR⁹ group, a C₂₋₆-alkenyl, or C₂₋₆-alkynyl group,
a straight-chain or branched C₁₋₆-alkyl group, wherein the hydrogen atoms of the straight-chain or branched C₁₋₆-alkyl group are optionally wholly or partly replaced by fluorine atoms, and wherein the straight-chain or branched C₁₋₆-alkyl group is optionally substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c} group, and additionally a methylene group adjacent to an abovementioned —NR^{8c} group is optionally replaced by a carbonyl group,
a phenyl or heteroaryl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
a phenyl-C₁₋₅-alkyl or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which are optionally substituted in the C₁₋₅-alkyl moiety by a hydroxy or a C₁₋₅-alkyloxy group, wherein the hydrogen atoms of the C₁₋₅-alkyloxy group are optionally wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group,
a 3- to 7-membered cycloalkyl, cycloalkyleneimino, cycloalkyl-C₁₋₅-alkyl, or cycloalkyleneimino-C₁₋₃-alkyl group, wherein in 4- to 7-membered cyclic groups in the cyclic moiety a methylene group is optionally replaced by a —N(R^{8c})— group, an oxygen or sulfur atom, or a —S(O) or —S(O)₂— group, or wherein in 4- to 7-membered cyclic groups in the cyclic moiety two adjacent methylene groups together are optionally replaced by a —C(O)N(R^{8b})— or —S(O)₂N(R^{8b})— group, or wherein in 6- to 7-membered cyclic groups in the cyclic moiety three adjacent methylene groups together are optionally replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyleneimino, cycloalkyl-C₁₋₅-alkyl, or cycloalkyleneimino-C₁₋₃-alkyl group as hereinbefore defined wherein two heteroatoms selected from oxygen and nitrogen are separated from one another by precisely one optionally substituted —CH₂— group, is excluded, wherein a 3- to 7-membered cycloalkyl, cycloalkyleneimino, cycloalkyl-C₁₋₅-alkyl, or cycloalkyleneimino-C₁₋₃-alkyl group as hereinbefore defined is optionally substituted at one or two —CH₂— groups by one or two C₁₋₃-alkyl groups in each case,
with the proviso that R⁴ and R⁵ are not both hydroxy or —OR⁹ groups, and wherein:
R⁹ is a straight-chain or branched C₁₋₆-alkyl group, wherein the hydrogen atoms of the straight-chain or branched C₁₋₆-alkyl group are optionally wholly or partly replaced by fluorine atoms, and wherein the straight-chain or branched C₁₋₆-alkyl group is optionally substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkyl-amino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c}— group, and additionally a methylene group adjacent to an abovementioned —NR^{8c}— group is optionally replaced by a carbonyl group, with the proviso that the replacement of hydrogen atoms of the first carbon atom of the straight-chain or branched C₁₋₆-alkyl group by substituents selected from O, S, or N is excluded,
a phenyl, heteroaryl, phenyl-C₁₋₅-alkyl, or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, a 3- to 7-membered cycloalkyl, cycloalkyl-C₁₋₅-alkyl, or cycloalkyleneimino-C₂₋₃-alkyl group, wherein in 4- to 7-membered cyclic groups in the cyclic moiety a methylene group is optionally replaced by a —N(R^{8c})— group, an oxygen or sulfur atom or a —S(O)— or —S(O)₂— group, or wherein in 4- to 7-membered cyclic groups in the cyclic moiety two adjacent methylene groups together are optionally replaced by a —C(O)N(R^{8b})— or —S(O)₂N(R^{8b})— group, or wherein in 6- to 7-membered cyclic groups in the cyclic moiety three adjacent methylene groups together are optionally replaced by a substituted —OC(O)N(R^{8b})— or —N(R^{8b})C(O)N(R^{8b})— or —N(R^{8b})S(O)₂N(R^{8b})— group, with the proviso that a 3- to 7-membered cycloalkyl, cycloalkyl-C₁₋₅-alkyl, or cycloalkyleneimino-C₂₋₃-alkyl group as hereinbefore defined wherein two heteroatoms selected from oxygen and nitrogen are separated from one another by precisely one optionally substituted —CH₂— group, is excluded, wherein a 3- to 7-membered cycloalkyl, cycloalkyl-C₁₋₅-alkyl, or cycloalkyleneimino-C₂₋₃-alkyl group as hereinbefore defined is optionally substituted at one or two —CH₂— groups by in each case one or two C₁₋₃-alkyl groups, or
R⁴ and R⁵ together with the carbon atom to which they are bound, form a C₃₋₈-cycloalkyl or C₃₋₈-cycloalkenyl group, wherein one of the methylene groups of a C₄₋₈-cycloalkyl group is optionally replaced by an oxygen or sulfur atom or a —N(R^{8c})—, carbonyl, sulfonyl, or sulfonyl group, and/or two directly adjacent methylene groups of a C₄₋₈-cycloalkyl group are together optionally replaced by a —C(O)N(R^{8b})— or —S(O)₂N(R^{8b})— group, and/or three directly adjacent methylene groups of a C₆₋₈-cycloalkyl group are together optionally replaced by an —OC(O)N(R^{8b})—, —N(R^{8b})C(O)N(R^{8b})—, or —N(R^{8b})S(O)₂N(R^{8b})— group, wherein one to three carbon atoms of a C₃₋₈-cycloalkyl group are optionally substituted independently of one another by in each case one or two identical or different halogen atoms, or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaninosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino groups, wherein 1 to 2 carbon atoms of a C₃₋₈-cycloalkenyl group are optionally substituted independently of one another by in each case a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, or C₃₋₆-cycloalkyleneiminosulfonyl group, and 1 to 2 carbon atoms of a C₄₋₈-cycloalkenyl group which are not bound to another carbon atom by a double bond, are optionally substituted independently of one another by a fluorine atom or a hydroxy, C₁₋₅-alkyloxy, C₁₋₅-alkylcarbonyloxy, C₁₋₅-alkylsulfanyl, C₁₋₅-alkylsulfonyl, amino, C₁₋₅-alkylamino, di-(C₁₋₅-alkyl)-amino, C₁₋₅-alkylcarbonylamino, C₁₋₅-alkyl-sulfonylamino, N—(C₁₋₅-alkylsulfonyl)-C₁₋₅-alkylamino, or C₃₋₆-cycloalkylcarbonylamino group, with the proviso that a C₃₋₈-cycloalkyl or C₃₋₈-cycloalkenyl group of this kind, formed from R⁴ and R⁵ together, wherein two heteroatoms in the cyclic group selected from oxygen and nitrogen are separated from one another by precisely one optionally substituted —CH₂— group, and/or wherein one or both methylene groups of the cyclic group which are directly connected to the carbon atom to which the groups R⁴ and R⁵ are bound are replaced by a heteroatom selected from O, N, and S, and/or wherein a substituent bound to the cyclic group, which is characterized in that a heteroatom selected from O, N, and S, and a halogen atom is bound directly to the cyclic group, is separated from another heteroatom selected from O, N, and S, with the exception of the sulfone group, by precisely one, optionally substituted, methylene group, and/or wherein two oxygen atoms are joined together directly, is excluded,
M is a —CH₂—, —CHR³—, —CR³R³— group, or a bond;
W is an oxygen or sulfur atom;
B is a thiophene ring of formula (III) 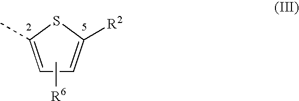
which is bound to the carbonyl group in formula (I) via the 2-position and which is substituted in the 5-position by R² and optionally additionally by R⁶, wherein:
R² is a fluorine, chlorine, bromine, or iodine atom, or a methoxy, C₁₋₂-alkyl or ethynyl group, and
R⁶ is a hydrogen, fluorine, chlorine, bromine, or iodine atom, or a C₁₋₂-alkyl or amino group,
wherein, unless stated otherwise, the term heteroaryl group means a monocyclic 5- or 6-membered heteroaryl group, wherein:
the 6-membered heteroaryl group contains one, two, or three nitrogen atoms, and
the 5-membered heteroaryl group contains an imino group optionally substituted by a C₁₋₃-alkyl group, or an oxygen or sulfur atom, or an imino group optionally substituted by a C₁₋₃-alkyl group, or an oxygen or sulfur atom and additionally a nitrogen atom, or an imino group optionally substituted by a C₁₋₃-alkyl group and two or three nitrogen atoms,
and moreover a phenyl ring optionally substituted by a fluorine, chlorine, or bromine atom, a C₁₋₃-alkyl, hydroxy, C₁₋₃-alkyloxy group, amino, C₁₋₃-alkylamino, di-(C₁₋₃-alkyl)-amino, or C₃₋₆-cycloalkyleneimino group are optionally fused to the abovementioned monocyclic heteroaryl groups via two adjacent carbon atoms,
and the bond is effected via a nitrogen atom or a carbon atom of the heterocyclic moiety or a fused-on phenyl ring,
wherein, unless stated otherwise, the term halogen atom means a fluorine, chlorine, bromine, or iodine atom,
wherein the alkyl, alkenyl, alkynyl and alkoxy groups contained in the previously mentioned definitions which have more than two carbon atoms are, unless stated otherwise, straight-chain or branched and the alkyl groups in the previously mentioned dialkylated groups are identical or different,
and the hydrogen atoms of the methyl or ethyl groups contained in the foregoing definitions, unless otherwise stated, are optionally wholly or partly replaced by fluorine atoms,
or a tautomer, enantiomer, or salt thereof.

2. The compound of formula (I) according to claim 1, wherein:
K¹ and K⁴ are each independently a —CH₂, —CHR^{7a}, —CR^{7b}R^{7c}, or a —C(O) group, wherein R^{7a}/R^{7b}/R^{7c} are each independently a fluorine atom, a hydroxy, C₁₋₅-alkoxy group, a C₁₋₅-alkyl group optionally substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂— group, or two groups R^{7b}/R^{7c} together with the cyclic carbon atom form a 3-, 4-, 5-, 6-, or 7-membered saturated carbocycle or a cyclopentene, cyclohexene, oxetan, tetrahydrofuran, tetrahydropyran ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides an N atom is optionally replaced by a —C(O)— group;
K² and K³ are each independently a —CH₂, —CHR^{8a}, —CR^{8b}R^{8c}, or a —C(O)— group, wherein R^{8a}/R^{8b}/R^{8c} are each independently a C₁₋₅-alkyl group optionally substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom form a 3-, 4-, 5-, 6-, or 7-membered saturated carbocycle or a cyclopentene, cyclohexene, oxetan, tetrahydrofuran, tetrahydropyran ring, wherein the methylene groups thereof are optionally substituted by one or two C₁₋₃-alkyl or —CF₃ groups are optionally substituted, and/or the methylene groups thereof, if they are not bound to a heteroatom, is optionally substituted by one or two fluorine atoms, and/or wherein a —CH₂— group besides a nitrogen atom is optionally replaced by a —C(O)— group, with the proviso that a heteroatom introduced by R^{8b} or R^{8c} cannot be separated from X in formula I by only one carbon atom,
or a tautomer, enantiomer, or salt thereof.

3. The compound of formula (I) according to one of claim 1 or 2, wherein:
K¹ and K⁴ are each independently a —CH₂—, —CHR^{7a}—, —CR^{7b}R^{7c}—, or a —C(O)— group, wherein R^{7a}/R^{7b}/7^{c} are each independently a fluorine atom, a hydroxy, C₁₋₅-alkoxy group, a C₁₋₅-alkyl group optionally substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom form a 3-, 4-, 5-, 6-, or 7-membered saturated carbocycle or a cyclopentene, cyclohexene, oxetan, tetrahydro furan, tetrahydropyran ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides an N atom is optionally replaced by a —C(O)— group;
K² and K³ are each independently a —CH₂—, —CHR^{8a}—, —CR^{8b}R^{8c}—, or a —C(O)— group, wherein R^{8a}/R^{8b}/R^{8c} are each independently a C₁₋₅-alkyl group optionally substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom form a 3-, 4-, 5-, 6-, or 7-membered saturated carbocycle or a cyclopentene, cyclohexene, oxetan, tetrahydrofuran, tetrahydropyran ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides a nitrogen atom is optionally replaced by a —C(O)— group, with the proviso that a heteroatom introduced by R^{8b} or R^{8c} are not be separated from X in formula I by only one carbon atom;
X is a —N(R¹)— group, wherein R¹ is a hydrogen atom or a C₁₋₅-alkyl, C₃₋₅-alkenyl-CH₂—, C₃₋₅-alkynyl-CH₂—, C₃₋₆-cycloalkyl, or C₄₋₆-cycloalkenyl group, wherein the methylene and methyl groups present in the groups mentioned previously are optionally additionally substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is a sulfur atom, a —C(R¹⁰)═N—, —N═C(R¹⁰), or —C(R¹⁰)═C(R¹¹)— group,
A² is a nitrogen atom or a ═C(R¹²)— group, wherein R¹⁰, R¹¹, and R¹² are each independently a hydrogen, fluorine, chlorine, or bromine atom, or a C₁₋₅-alkyl, —CF₃, cyano, carboxy, C₁₋₅-alkoxycarbonyl, hydroxy, C₁₋₃-alkoxy, CF₃O—, CHF₂O—, CH₂FO— group;
L is a substituted ring system of formula (IIa) 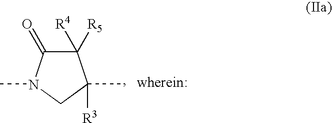
R³ is a hydrogen atom;
R⁴ and R⁵ are each independently a hydrogen atom, a hydroxy group, an —OR⁹ group, a C₂₋₆-alkenyl, or C₂₋₆-alkynyl group,
a straight-chain or branched C₁₋₆-alkyl group, wherein the hydrogen atoms of the straight-chain or branched C₁₋₆-alkyl group are optionally wholly or partly replaced by fluorine atoms, and wherein the straight-chain or branched C₁₋₆-alkyl group is optionally substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-allylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneimnocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c} group, and additionally a methylene group adjacent to an abovementioned —NR^{8c} group is optionally replaced by a carbonyl group,
a phenyl or heteroaryl group, each optionally be mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
a phenyl-C₁₋₅-alkyl or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which are optionally substituted in the C₁₋₅-alkyl moiety by a hydroxy or a C₁₋₅-alkyloxy group, wherein the hydrogen atoms of the C₁₋₅-alkyloxy group are optionally wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group,
a 3- to 7-membered cycloalkyl, cycloalkyleneimino, cycloalkyl-C₁₋₅-alkyl, or cycloalkyleneimino-C₁₋₃-alkyl group, wherein in 4- to 7-membered cyclic groups in the cyclic moiety a methylene group is optionally replaced by a —N(R^{8c})— group, an oxygen or sulfur atom, or a —S(O)— or —S(O)₂— group, or wherein in 4- to 7-membered cyclic groups in the cyclic moiety two adjacent methylene groups together are optionally replaced by a —C(O)N(R^{8b})— or —S(O)₂N(R^{8b})— group, or wherein in 6- to 7-membered cyclic groups in the cyclic moiety three adjacent methylene groups together are optionally replaced by a substituted —OC(O)N(R^{8b})— or —N(R^{8b})C(O)N(R^{8b})— or —N(R^{8b})S(O)₂N(R^{8b})— group, with the proviso that a defined 3- to 7-membered cycloalkyl, cycloalkyleneimino, cycloalkyl-C₁₋₅-alkyl, or cycloalkyleneimino-C₁₋₃-alkyl group as hereinbefore wherein two heteroatoms selected from oxygen and nitrogen are separated from one another by precisely one optionally substituted —CH₂— group, is excluded, wherein a 3- to 7-membered cycloalkyl, cycloalkyleneimino, cycloalkyl-C₁₋₅-alkyl, or cycloalkyleneimino-C₁₋₃-alkyl group as hereinbefore defined are optionally substituted at one or two —CH₂— groups by one or two C₁₋₃-alkyl groups in each case, and
R⁹ is a straight-chain or branched C₁₋₆-alkyl group, wherein the hydrogen atoms of the straight-chain or branched C₁₋₆-alkyl group are optionally wholly or partly replaced by fluorine atoms, and wherein the straight-chain or branched C₁₋₆-alkyl group is optionally substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c}— group, and additionally a methylene group adjacent to an abovementioned —NR^{8c}— group is optionally replaced by a carbonyl group, with the proviso that the replacement of hydrogen atoms of the first carbon atom of the straight-chain or branched C₁₋₆-alkyl group by substituents selected from O, N, or S is excluded,
a phenyl, heteroaryl, phenyl-C₁₋₅-alkyl, or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
a 3- to 7-membered cycloalkyl, cycloalkyl-C₁₋₅-alkyl, or cycloalkyleneimino-C₂₋₃-alkyl group, wherein in 4- to 7-membered cyclic groups in the cyclic moiety a methylene group is optionally replaced by a —N(R^{8c}) group, an oxygen or sulfur atom or a —S(O)— or —S(O)₂— group, or wherein in 4- to 7-membered cyclic groups in the cyclic moiety two adjacent methylene groups together are optionally replaced by a —C(O)N(R^{8b})— or —S(O)₂N(R^{8b})— group, or wherein in 6- to 7-membered cyclic groups in the cyclic moiety three adjacent methylene groups together are optionally replaced by a substituted —OC(O)N(R^{8b})— or N(R^{8b})C(O)N(R^{8b})— or —N(R^{8b})S(O)₂N(R^{8b})— group, with the proviso that a 3- to 7-membered cycloalkyl, cycloalkyl-C₁₋₅-alkyl, or cycloalkyleneimino-C₂₋₃-alkyl group as hereinbefore defined wherein two heteroatoms selected from oxygen and nitrogen are separated from one another by precisely one optionally substituted —CH₂— group, is excluded, wherein a 3- to 7-membered cycloalkyl, cycloalkyl-C₁₋₅-alkyl, or cycloalkyleneimino-C₂₋₃-alkyl group as hereinbefore defined are optionally substituted at one or two —CH₂— groups by one or two C₁₋₃-alkyl groups in each case, and
R⁶ is a hydrogen atom,
or a tautomer, enantiomer, or salt thereof.

4. The compound of formula (I) according to one of claims 1 to 3, wherein:
K¹ and K⁴ are each independently a —CH₂—, —CHR^{7a}, —CR^{7b}R^{7c}, or a —C(O)— group, wherein R^{7a}/R^{7b}/R^{7c} are each independently a fluorine atom, a hydroxy, C₁₋₅-alkoxy group, a C₁₋₅-alkyl group optionally substituted by one to three fluorine atoms, a hydroxy-C₁₋₅-alkyl, C₁₋₅-alkoxy-C₁₋₅-alkyl group, wherein the two groups R^{7b}/R^{7c} cannot both simultaneously be bound to the cyclic carbon atom via a heteroatom, except where —C(R^{7b}R^{7c})— corresponds to a —CF₂ group, or two groups R^{7b}/R^{7c} together with the cyclic carbon atom form a 3-, 4-, 5-, 6-, or 7-membered saturated carbocycle or a cyclopentene, cyclohexene, oxetan, tetrahydroffuran, tetrahydropyran ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides an N atom is optionally replaced by a —C(O)— group;
K² and K³ are each independently a —CH₂—, —CHR^{8a}—, —CR^{8b}R^{8c}, or a —C(O)— group, wherein R^{8a}/R^{8b}/R⁸ are each independently a C₁₋₅-alkyl group optionally substituted by one to three fluorine atoms, a hydroxy-C₁₋₅-alkyl, C₁₋₅-alkoxy-C₁₋₅-alkyl group, or two groups R^{8b}/R^{8c} together with the cyclic carbon atom form a 3-, 4-, 5-, 6-, or 7-membered saturated carbocycle or a cyclopentene, cyclohexene, oxetan, tetrahydrofuiran, tetrahydropyran ring, wherein the methylene groups thereof are optionally substituted by one or two C₁₋₃-alkyl or —CF₃ groups, and/or the methylene groups thereof, if they are not bound to a heteroatom, are optionally substituted by one or two fluorine atoms, and/or wherein a —CH₂— group besides a nitrogen atom is optionally replaced by a —C(O)— group, with the proviso that a heteroatom introduced by R^{8b} or R^{8c} cannot be separated from X in formula I by only one carbon atom;
X is a —N(R¹)— group, wherein R¹ is a hydrogen atom or a C₁₋₅-alkyl, C₃₋₅-alkenyl-CH₂, C₃₋₅-alkynyl-CH₂, C₃₋₆-cycloalkyl, or C₄₋₆-cycloalkenyl group, wherein the methylene and methyl groups present in the groups mentioned previously are optionally additionally substituted by a C₁₋₃-alkyl, carboxy, C₁₋₅-alkylcarboxycarbonyl group, or by a hydroxy, C₁₋₅-alkoxy, amino, C₁₋₅-alkylamino, C₁₋₅-dialkylamino, or C₄₋₇-cycloalkyleneimino group provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is a sulfur atom, a —C(R¹⁰)═N—, —N═C(R¹⁰)—, or —C(R¹⁰)═C(R¹¹)— group,
A² is a nitrogen atom or a ═C(R¹²)— group, wherein R¹⁰, R¹¹, and R¹² are each independently a hydrogen, fluorine, chlorine, bromine atom, or a C₁₋₅-alkyl, —CF₃, cyano, carboxy, C₁₋₅-alkoxycarbonyl, hydroxy, C₁₋₃-alkoxy, CF₃O—, CHF₂O—, or CH₂FO— group;
L is a substituted ring system of formula (IIa) 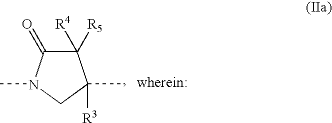
R³ is a hydrogen atom;
R⁴ is a hydrogen atom, a straight-chain or branched C₁₋₄-alkyl group, wherein the hydrogen atoms of the straight-chain or branched C₁₋₄-alkyl group are optionally wholly or partly replaced by fluorine atoms, and which are optionally substituted by a C₁₋₃-alkoxy group, wherein the hydrogen atoms of the C₁₋₃-alkoxy group are optionally wholly or partly replaced by fluorine atoms;
R⁵ is a hydrogen atom, a hydroxy group, an —OR⁹ group, a C₂₋₄-alkenyl, or C₂₋₄-alkynyl group,
a straight-chain or branched C₁₋₄-alkyl group, wherein the hydrogen atoms of the straight-chain or branched C₁₋₄-alkyl group are optionally wholly or partly replaced by fluorine atoms, and wherein the straight-chain or branched C₁₋₄-alkyl group is optionally substituted by a C₃₋₅-cycloalkyl, nitrile, hydroxy, C₁₋₅-alkyloxy group, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulfanyl, C₁₋₅-alkylsulfonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulfonyl, C₁₋₅-alkylaminosulfonyl, di-(C₁₋₅-alkyl)-aminosulfonyl, C₃₋₆-cycloalkyleneiminosulfonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₄-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c} group, and additionally a methylene group adjacent to an abovementioned —NR^{8c} group is optionally replaced by a carbonyl group,
a phenyl or heteroaryl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups,
a phenyl-C₁₋₅-alkyl or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups, and which are optionally substituted in the C₁₋₅-alkyl moiety by a hydroxy or a C₁₋₅-alkyloxy group, wherein the hydrogen atoms of the C₁₋₅-alkyloxy group are optionally wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, or a C₁₋₅-alkyloxycarbonyl-C₁₋₅-alkyloxy group, and wherein:
R⁹ is a straight-chain or branched C₁₋₄-alkyl group, wherein the hydrogen atoms of the straight-chain or branched C₁₋₄-alkyl group are optionally wholly or partly replaced by fluorine atoms, and wherein the straight-chain or branched C₁₋₄-alkyl group is optionally substituted by a C₃₋₅-cycloalkyl group, hydroxy, C₁₋₅-alkyloxy, allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, amino, C₁₋₅-alkylamino, di-(C₁₋₅-alkyl)-amino, C₁₋₅-alkylcarbonylamino, C₁₋₅-alkylsulfonylamino, N—(C₁₋₅-alkylsulfonyl)-C₁₋₅-alkylamino, or C₃₋₆-cycloalkylcarbonylamino group, wherein the hydrogen atoms of the C₁₋₅-alkyloxy group are optionally wholly or partly replaced by fluorine atoms, and in the 6- to 7-membered cyclic groups of the C₃₋₆-cycloalkyleneiminocarbonyl group in the cyclic moiety a methylene group in the 4-position of a 6- or 7-membered cycloalkyleneimino group is optionally replaced by an oxygen or sulfur atom, by a carbonyl, sulfinyl, sulfonyl, or —NR^{8c}— group, and additionally a methylene group adjacent to an abovementioned —NR^{8c}— group is optionally replaced by a carbonyl group, with the proviso that the replacement of hydrogen atoms of the first carbon atom of the straight-chain or branched C₁₋₆-alkyl group by substituents selected from O, N, or S is excluded,
a phenyl, heteroaryl, phenyl-C₁₋₅-alkyl, or heteroaryl-C₁₋₅-alkyl group, which are optionally mono- to tri-substituted in the phenyl or heteroaryl moiety by identical or different substituents selected from halogen atoms, C₁₋₅-alkyl, di-(C₁₋₅-alkyl)-amino, hydroxy, C₁₋₅-alkyloxy, mono-, di-, or trifluoromethoxy, carboxy-, and C₁₋₅-alkyloxycarbonyl groups;
M is a —CH₂— group or a bond;
W is an oxygen atom;
B is a thiophene ring according to formula (III) 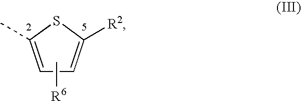
which is bound to the carbonyl group in formula (I) via the 2-position and which is substituted in the 5-position by R² and optionally additionally by R⁶, wherein:
R² is a fluorine, chlorine, bromine, or iodine atom, or a methoxy, C₁₋₂-alkyl, or ethynyl group, and
R⁶ is a hydrogen atom,
or a tautomer, enantiomer, or salt thereof

5. The compound of formula (I) according to one of claims 1 to 4, wherein:
K¹ and K⁴ are each independently a —CH₂, —CHR^{7a}, —CR^{7b}R^{7c}, or a —C(O) group, wherein R^{7a}/R^{7b}/R^{7c} are each independently a C₁₋₂-alkyl group optionally substituted by one to three fluorine atoms;
K² and K³ are each a —CH₂— group;
X is a —N(R¹)— group, wherein R¹ is a hydrogen atom, or a C₁₋₅-alkyl or C₃₋₄-cycloalkyl group, wherein the methylene and methyl groups present in the groups mentioned previously are optionally additionally substituted by a hydroxy group, provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is a sulfur atom or —C(R¹⁰)═C(R¹¹)— group,
A² is a nitrogen atom or a ═C(R¹²)— group, wherein R¹⁰, R¹¹, and R¹² are each independently a hydrogen, fluorine, or chlorine atom, or a C₁₋₅-alkyl, —CF₃, methoxy, CF₃O—, CHF₂O—, CH₂FO— group;
L is a substituted ring system of formula (IIa) 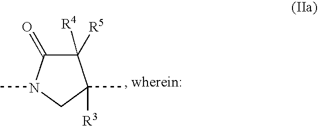
R³ and R⁴ are each a hydrogen atom;
R⁵ is a hydrogen atom or a C₂₋₄-alkenyl or C₂₋₄-alkynyl group,
a straight-chain or branched C₁₋₄-alkyl group, wherein the hydrogen atoms of the straight-chain or branched C₁₋₄-alkyl group are optionally wholly or partly replaced by fluorine atoms, and wherein the straight-chain or branched C₁₋₄-alkyl group is optionally substituted by a hydroxy, C₁₋₅-alkyloxy group or a di-(C₁₋₅-alkyl)-aminocarbonyl group, wherein the hydrogen atoms of the C₁₋₅-alkyloxy group are optionally wholly or partly replaced by fluorine atoms,
a phenyl-C₁₋₃-alkyl or heteroaryl-C₁₋₃-alkyl group;
M is a —CH₂— group or a bond;
W is an oxygen atom;
B is a thiophene ring according to formula (III) 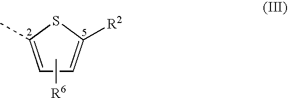
which is bound to the carbonyl group in formula (I) via the 2-position and which is substituted in the 5-position by R² and optionally additionally by R⁶, wherein:
R² is a chlorine or bromine atom, or an ethynyl group; and
R⁶ is a hydrogen atom,
or a tautomer, enantiomer, or salt thereof.

6. The compound of formula (I) according to one of claims 1 to 5, wherein:
K¹ and K⁴ are each independently a —CH₂—, —CHR^{7a}, —CR^{7b}R^{7c}, or a —C(O)— group, wherein R^{7a}/R^{7b}/R^{7c} are each independently a C₁₋₂-alkyl group optionally substituted by one to three fluorine atoms;
K² and K³ are each a —CH₂— group;
X is a —N(R¹)— group, wherein R¹ is a hydrogen atom, or a C₁₋₅-alkyl or C₃₋₄-cycloalkyl group, wherein the methylene and methyl groups present in the groups mentioned previously are optionally additionally substituted by a hydroxy group, provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is a sulfur atom, and
A² is a nitrogen atom,
or a tautomer, enantiomer, or salt thereof

7. The compound of formula (I) according to one of claims 1 to 6, wherein:
K¹ and K⁴ are each independently a —CH₂—, —CHR^{7a}—, CR^{7b}R^{7c}—, or a —C(O)— group, wherein R^{7a}/R^{7b}/R⁷ are each independently a C₁₋₂-alkyl group optionally substituted by one to three fluorine atoms;
K² and K³ are each a —CH₂— group;
X is a —N(R¹)— group, wherein R¹ is a hydrogen atom or a C₁₋₅-alkyl or C₃₋₄-cycloalkyl group, wherein the methylene and methyl groups present in the groups mentioned previously are optionally additionally substituted by a hydroxy group, provided that the methylene or methyl groups are not directly bound to a heteroatom selected from O, N, or S, and/or one to three hydrogen atoms are optionally replaced by fluorine atoms, provided that the methylene or methyl groups are not directly bound to a heteroatom selected from O, N, or S;
A¹ is a —C(R¹⁰)═C(R¹¹)— group, and
A² is a ═C(R¹²)— group, wherein R¹⁰, R¹¹, and R¹² are each independently a hydrogen, fluorine, or chlorine atom, or a C₁₋₅-alkyl, —CF₃, methoxy, CF₃O—, CHF₂O—, or CH₂FO— group,
or a tautomer, enantiomer, or salt thereof.

8. A compound of formula (I) according to one of claims 1 to 7, wherein M is a bond, or a tautomer, enantiomer, or salt thereof.

9. A physiologically acceptable salt of the compound according to one of claims 1 to 8.

10. A pharmaceutical composition containing the compound according to one of claims 1 to 8 or a physiologically acceptable salt according to claim 9 and one or more inert carriers and/or diluents.
